(19) United States
(12) Patent Application Publication
(10) Pub. No.: US 2025/0164510 A1
(43) Pub. Date: May 22, 2025

(54) USE OF GAMMA PRIME FIBRINOGEN AS A BIOMARKER IN THE ASSESSMENT OF COVID-19 INFECTIONS AND PROGNOSIS OF SEVERE DISEASE

(71) Applicant: Gamma Diagnostics Inc., Portland, OR (US)

(72) Inventors: Cn RAMCHAND, Tharamani Chennai (IN); David H. FARRELL, Tualatin, OR (US); Steven C. KAZMIERCZAK, Portland, OR (US); William MESSER, Portland, OR (US); Akram KHAN, Portland, OR (US); Ajay Sasikumar NAIR, Yokine Western Australia (AU)

(73) Assignee: Gamma Diagnostics Inc., Portland, OR (US)

(21) Appl. No.: 18/841,731

(22) PCT Filed: Jul. 5, 2022
(86) PCT No.: PCT/US2022/073412

§ 371 Date: Aug. 27, 2024

Related U.S. Application Data
(60) Provisional application No. 63/268,605, filed on Feb. 27, 2022.

(51) Int. Cl. G01N 33/86; G01N 33/68

(52) U.S. Cl. CPC G01N 33/86; G01N 33/6869; G01N 33/6893; G01N 2333/4737; G01N 2333/5412; G01N 2333/75; G01N 2800/26

(57) ABSTRACT

A method of assessing a COVID-19 infection in a person, the method comprising analysing the concentration or levels of one or more biomarkers in a biological sample from a person, wherein the one or more biomarkers comprises γ′ fibrinogen, and wherein concentration or levels of γ′ fibrinogen are elevated in a biological sample from a person infected with COVID-19 and can be used for predicting COVID-19 disease severity and/or for making a prognosis of severe COVID-19 disease in a person infected with COVID-19.

### FIELD OF THE INVENTION

The present invention relates to methods and uses for assessment of COVID-19 infection and prognosis of disease severity upon elevation of biomarkers comprising at least γ′ fibrinogen in the blood plasma of a person infected with COVID-19.

### BACKGROUND

The following discussion of the background art is intended to facilitate an understanding of the present invention only. It should be appreciated that the discussion is not an acknowledgement or admission that any of the material referred to was part of the common general knowledge as at the priority date of the application.

Coronavirus disease 2019 (COVID-19) is a viral respiratory disease and causes inflammation of the mucosal membrane. This leads to alveolar damage and eventually pneumonia. It is caused by SARS-COV-2, commonly known as novel coronavirus, a positive-sense single-stranded RNA virus. Coronaviruses have previously been reported to cause severe acute respiratory syndrome (SARS) and Middle East respiratory syndrome (MERS).

Mild symptoms experienced by persons infected with COVID-19 have included fever, dry cough, tiredness, sore throat, diarrhea, headache, conjunctivitis, nasal congestion, body aches and pains, fatigue, loss of smell and taste, a rash on skin or discoloration of fingers or toes. However, more severe acute respiratory syndrome coronavirus 2 (SARS-COV-2)-induced infection, the cause of COVID-19, is characterized by unprecedented clinical pathologies commonly requiring the need for mechanical ventilation or extracorporeal membrane oxygenation (ECMO) (often referred to as requiring ‘ventilation’) of an infected person, and in many cases resulting in their death.

There have already been many investigations into COVID-19 which amongst other effects, has been shown to cause inflammation, coagulation, and altered haemostasis in an infected person. The associated coagulation disorders were found to be one of the leading causes of death of persons infected with COVID-19. Phenotypic vascular characteristics are strongly associated with various coagulopathies that may result in either bleeding and thrombocytopenia or hypercoagulation and thrombosis. Growing evidence documents elevated pro inflammatory cytokines (e.g. interleukin-6 (IL-6)) in patients with COVID-19, which is thought to lead to derangement in vascular function and blood composition. Accordingly, increased circulating von Willebrand factor (VWF), thrombomodulin (TM) and factor VIII suggest COVID-19 induces endothelial activation and elevated C-reactive protein (CRP) and fibrinogen indicate the infection activates the acute phase response.

Increased D-dimer and prothrombin time (PT) have also been reported in severe COVID-19 patients which indicates the formation and degradation of clots. Abnormal coagulation parameters are associated with poor prognosis in patients with novel coronavirus pneumonia.

In addition, thrombin, fibrin, and plasmin have central roles not only in thrombosis but also as effectors in infection and host responses, suggesting dysregulation of these pathways may contribute in multiple ways to the pathophysiology of SARS-COV-2infection. In COVID-19 patients, activation of coagulation-related factors and the immune system leads to blood clots in the vessels. Activation of coagulation in the pulmonary artery causes pulmonary embolism. These factors cause changes in coagulation-related biomarkers and include, amongst various others, D-dimer, CRP, PT and partial thromboplastin time (PTT), IL-6, fibrinogen, platelet count, lactate dehydrogenase (LDH), neutrophils and lymphocytes.

While there are already various biomarkers which have already been identified for assessing different aspects of COVID-19 infection in a person, there is always scope for additional biomarkers for assisting in the determination of infection, diagnosis of host responses, prognosis including predicting disease severity, therapeutic efficacy, amongst other potential uses.

### SUMMARY OF INVENTION

Fibrinogen is a glycoprotein which is converted enzymatically to a protein called fibrin during tissue injury to form fibrin clots that primarily function to occlude blood vessels to stop bleeding. Fibrinogen is primarily made and secreted into the blood by hepatocytes. The final structure consists of two trimers with each trimer composed of three different polypeptide chains:
1. the fibrinogen alpha chain (also termed the Aα or α chain) encoded by the FGA gene.
2. the fibrinogen beta chain (also termed the Bβ or β chain) encoded by the FGB gene.
3. the fibrinogen gamma chain (also termed the γ chain) encoded by the FGG gene.

The common form of fibrinogen in blood is termed as γA and constitutes 85-90% of total fibrinogen. Alternative splicing of the FGG gene produces a minor isoform of the γ chain termed as the γ′ (gamma prime) which replaces 8-10% of circulating fibrinogen. The γ′ isoform arises from alternative mRNA splicing in intron 9, resulting in the loss of exon 10 and retention of a part of intron 9 in the mRNA. As a result, the carboxy terminus is translated with an additional different 20-amino acid sequence VRPEHPAETEYDSLYPEDDL (γ′ 408-427) which is not present in the common γA isoform.

γ′ fibrinogen has several biochemical and biophysical properties that distinguish it from the more common γA (γA 408-411; AGDV) isoform. Clots made from fibrinogen containing γ′ chains in the presence of factor XIII are highly resistant to fibrinolysis. In addition, the γ′ chain contains a binding site for thrombin which protects it from inhibition by antithrombin and heparin. As a result, clots made from γ′ fibrinogen have been reported to have an altered clot architecture and has emerged as a risk factor for cardiovascular diseases. The inflammation/coagulation interface is involved in a variety of disease processes.

Inventors made the discoveries that:

The rare γ′ isoform, γ′ fibrinogen ('GPF') is found at elevated concentrations in the plasma of a person (or animal) infected with COVID-19 when compared to persons that are free from COVID-19 infection (COVID-19-free subjects).

γ′ fibrinogen levels correlate to SpO₂ levels and associated disease severity in a person infected with COVID-19.

The concentration of γ′ fibrinogen in the plasma of a person infected with COVID-19 may be used as a prognostic indicator to assess the future likely severity of COVID-19 disease for the person.

In a first aspect, the present invention provides a method of assessing a COVID-19 infection in a person, the method comprising analysing the concentration or levels of one or more biomarkers in a biological sample from a person, wherein the one or more biomarkers comprises γ′ fibrinogen.

In an embodiment of a method according to the invention, analysing the concentration or levels of one or more biomarkers in a biological sample from a person, wherein the one or more biomarkers comprises γ′ fibrinogen, comprises detecting and quantifying the amount of the one or more biomarkers in a blood sample from the person.

In an embodiment of a method according to the invention, the concentration or levels of γ′ fibrinogen in a biological sample from a person infected with COVID-19 are elevated when compared to a biological sample taken from the person when the person was not infected with COVID-19 or when compared to biological samples taken from one or more other persons not infected with COVID-19.

In an embodiment of a method according to the invention, the concentration or levels of γ′ fibrinogen is elevated above a reference or cut-off value that is preferably greater than between approximately 40 mg/dL to 50 mg/dL, more preferably greater than approximately 46 mg/dL in a biological sample from a person infected with COVID-19.

In an embodiment of a method according to the invention, analysing the concentration or levels of one or more biomarkers in a biological sample from a person, wherein the one or more biomarkers comprises γ′ fibrinogen, is used for predicting COVID-19 disease severity and/or for making a prognosis of severe COVID-19 disease in a person infected with COVID-19.

In an embodiment of a method according to the invention, a concentration of γ′ fibrinogen of between approximately 40 mg/dl to 70 mg/dL, more preferably between approximately 46 mg/dL and 67 mg/dL in a biological sample from a person indicates a mild to moderate COVID-19 infection in the person.

In an embodiment of a method according to the invention, a concentration of γ′ fibrinogen is elevated above a reference or cut-off value that is preferably approximately 60 mg/dL or greater, more preferably approximately 67 mg/dL or greater in a biological sample from a person indicates a severe COVID-19 infection in the person.

In an embodiment of a method according to the invention, a concentration of γ′ fibrinogen of between approximately 40 mg/dL to 70 mg/dL, more preferably between approximately 46 mg/dL and 67 mg/dL in the biological sample from the person indicates a mild to moderate COVID-19 infection in the person corresponding to a SpO₂ of above approximately 91% in the person.

In an embodiment of a method according to the invention, a concentration of γ′ fibrinogen of approximately 60 mg/dL or greater, more preferably approximately 67 mg/dL or greater in the biological sample from the person indicates a severe COVID-19 infection in the person corresponding to a SpO₂ of below approximately 91% in the person.

In an embodiment of a method according to the invention, an assay is used in analysing the concentration or levels of the one or more biomarkers in a biological sample from a person, wherein the one or more biomarkers comprises γ′ fibrinogen. The assay preferably comprises an ELISA.

The steps of the ELISA preferably comprise:
adding a sample to one or more assay plate wells coated with monoclonal capture antibody to capture the one or more biomarkers in the sample;
adding to the one or more assay plate wells enzyme-labelled polyclonal anti-fibrinogen detection antibody to the one or more biomarkers which binds to the captured one or more biomarkers;
adding a substrate for the enzyme-labelled polyclonal detection antibody to the one or more biomarkers; and
detecting a coloured end product in the one or more assay plate wells using a spectrophotometer for calculating the concentration or levels of the one or more biomarkers in the sample, wherein the one or more biomarkers comprises γ′ fibrinogen.

In an embodiment of a method according to the invention, the one or more biomarkers further comprise any one or more selected from the group comprising: D-Dimer, ferritin, CRP, and IL-6.

For the purposes of describing the invention, COVID-19 disease severity or severe COVID-19 disease comprises ICU admission and/or ventilation for the COVID-19 infected person, and/or a SpO₂ of below approximately 91% in the COVID-19 infected person.

The invention further provides a method of treating a person infected with COVID-19 according to the severity of their COVID-19 infection as determined using a method of assessing a COVID-19 infection in a person as described herein.

The invention further provides a method for prognosis of COVID-19 disease severity in a person comprising a method of assessing a COVID-19 infection in a person as described herein.

The invention further provides a method for assessing a COVID-19 infection in a person, the method comprising testing of a biological sample from the patient with a reagent for the detection of one or more biomarkers, wherein the one or more biomarkers comprises γ′ fibrinogen as herein described.

The invention further provides a method of detecting the concentration or levels of one or more biomarkers in a biological sample from a person infected with COVID-19 infection using an assay to make a prognosis on the severity of COVID-19 disease progression in the person, and wherein the one or more biomarkers comprises γ′ fibrinogen as described herein.

The invention further provides a method of treating a person infected with COVID-19 following a prognosis of severe COVID-19-related disease from analysing the concentration or levels of one or more biomarkers in a biological sample from the person, wherein the one or more biomarkers comprises γ′ fibrinogen as herein described.

The invention further provides a screening method to assess COVID-19 patients for severe disease progression using methods as described herein.

The invention further provides a kit for diagnosing, monitoring, and/or forming a prognosis of disease severity of COVID-19 in a person comprising an assay as described herein.

In a second aspect, the present invention provides the use of one or more biomarkers in assessing a COVID-19 infection in a person comprising analysing the concentration or levels of the one or more biomarkers in a biological sample from the person, wherein the one or more biomarkers comprises γ′ fibrinogen.

In an embodiment of a use according to the invention, analysing the concentration or levels of the one or more biomarkers in the biological sample from the person, wherein the one or more biomarkers comprises γ′ fibrinogen, comprises detecting and quantifying the amount of the one or more biomarkers in a blood sample from the person.

In an embodiment of a use according to the invention, concentration or levels of γ′ fibrinogen in a biological sample from a person infected with COVID-19 are elevated when compared to a biological sample taken from the person when the person was not infected with COVID-19 or when compared to biological samples taken from one or more other persons not infected with COVID-19.

In an embodiment of a use according to the invention, concentration or levels of γ′ fibrinogen are greater than between approximately 40 mg/dl to 50 mg/dl, more preferably greater than approximately 46 mg/dl in a biological sample from a person infected with COVID-19.

In an embodiment of a use according to the invention, wherein analysing the concentration or levels of one or more biomarkers in a biological sample from a person, wherein the one or more biomarkers comprises γ′ fibrinogen, is used for predicting COVID-19 disease severity and/or for making a prognosis of severe COVID-19 disease in a person infected with COVID-19.

In an embodiment of a use according to the invention, wherein a concentration of γ′ fibrinogen of between approximately 40 mg/dL to 70 mg/dL, more preferably between approximately 46 mg/dl and 67 mg/dL in a biological sample from a person indicates a mild to moderate COVID-19 infection in the person.

In an embodiment of a use according to the invention, a concentration of γ′ fibrinogen of approximately 60 mg/dL or greater, more preferably approximately 67 mg/dL or greater in a biological sample from a person indicates a severe COVID-19 infection in the person.

In an embodiment of a use according to the invention, a concentration of γ′ fibrinogen of between approximately 40 mg/dL to 70 mg/dL, more preferably between approximately 46 mg/dl and 67 mg/dL in the biological sample from the person indicates a mild to moderate COVID-19 infection in the person corresponding to a SpO₂ of above approximately 91% in the person.

In an embodiment of a use according to the invention, a concentration of γ′ fibrinogen of approximately 60 mg/dL or greater, more preferably approximately 67 mg/dL or greater in the biological sample from the person indicates a severe COVID-19 infection in the person corresponding to a SpO₂ of below approximately 91% in the person.

In an embodiment of a use according to the invention, an assay is used in analysing the concentration or levels of the one or more biomarkers in a biological sample from a person, wherein the one or more biomarkers comprises γ′ fibrinogen.

In an embodiment of a use according to the invention, the one or more biomarkers further comprise any one or more selected from the group comprising: D-Dimer, ferritin, CRP, and IL-6.

In an embodiment of a use according to the invention, COVID-19 disease severity or severe COVID-19 disease comprises ICU admission and/or ventilation for the COVID-19 infected person, and/or a SpO₂ of below approximately 91% in the COVID-19 infected person.

The invention further provides the use of one or more biomarkers for predicting disease severity in a person infected with COVID-19, comprising analysing the concentration or levels of the one or more biomarkers in a biological sample from the person, wherein the one or more biomarkers comprises γ′ fibrinogen as herein described.

The invention further provides the use of one or more biomarkers in a screening method for assessment and prognosis of an increased likelihood of a COVID-19-infected person developing severe disease, comprising analysing the concentration or levels of the one or more biomarkers in a biological sample from the person, wherein the one or more biomarkers comprises γ′ fibrinogen as herein described.

In an embodiment of the invention, the biological sample comprises one or more sample from the person selected from the group comprising: whole blood, blood fraction, and/or plasma. More preferably, the biological sample comprises blood plasma.

In an embodiment of the invention, the invention provides use of γ′ fibrinogen as a biomarker of COVID-19 infection in a person, and preferably when the COVID-19 infection leads to severe disease in the person.

In an embodiment of the invention, the invention provides use of γ′ fibrinogen as a biomarker to predict disease severity in a person infected with COVID-19.

In an embodiment of the invention, the invention provides use of γ′ fibrinogen as a prognostic indicator of severe disease in a person infected with COVID-19.

In an embodiment of the invention, the invention provides use of γ′ fibrinogen, D-dimer, and ferritin as biomarkers to predict disease severity in a person infected with COVID-19. A higher concentration or levels of γ′ fibrinogen, D-dimer, and ferritin is preferably an indicator of potential for increased disease severity.

In an embodiment of the invention, the invention provides use of γ′ fibrinogen in the manufacture of an assay for assessing and prognosis of disease and severe disease in a COVID-19-infected person.

In an embodiment of the invention, the invention provides use of γ′ fibrinogen in the manufacture of a prognostic, diagnostic, or monitoring tool for the assessment of a COVID-19 infection in a person or severe disease in a COVID-19 infected person.

### BRIEF DESCRIPTION OF DRAWINGS

In order to provide a better understanding, embodiments of the present invention will be described, by way of example only, with reference to the accompanying drawings, in which:

FIG. 1. (A) Bar chart comparing concentration of γ′ fibrinogen (GPF) between COVID-19-free subjects and COVID-19 patients, which was calculated from tabulated raw data from (B) the persons infected with COVID-19, and (C) the COVID-free subjects.

FIG. 2. (A) Bar chart comparing concentration of D-dimer between COVID-19-free subjects and COVID-19 patients, which was calculated from tabulated raw data from (B) the persons infected with COVID-19, and (C) the COVID-free subjects.

FIG. 3. (A) Bar chart comparing concentration of ferritin between COVID-19-free subjects and COVID-19 patients, which was calculated from tabulated raw data from (B) the persons infected with COVID-19, and (C) the COVID-free subjects.

FIG. 4. (A) Bar chart comparing concentration of CRP between COVID-19-free subjects and COVID-19 patients, which was calculated from tabulated raw data from (B) the persons infected with COVID-19, and (C) the COVID-free subjects.

FIG. 5. (A) Bar chart comparing concentration of IL-6 between COVID-19-free subjects and COVID-19 patients, which was calculated from tabulated raw data from (B) the persons infected with COVID-19, and (C) the COVID-free subjects.

FIG. 6. (A) Bar chart comparing concentration of LDH between COVID-19-free subjects and COVID-19 patients, which was calculated from tabulated raw data from (B) the persons infected with COVID-19, and (C) the COVID-free subjects.

FIG. 7. Table of raw data from (A) the persons infected with COVID-19, and (B) the COVID-free subjects, with concentrations for total fibrinogen.

FIG. 8. Table of comorbidity data for the persons infected with COVID-19.

FIG. 9. A ROC curve for GFP determined from COVID-19-free subjects and COVID-19 patients, and (B) showing a cut-point maximising both sensitivity and specificity using Youden's index.

FIG. 10. A ROC curve showing a logistic regression calculated using results for GPF, D-dimer, and ferritin from COVID-19-free subjects and COVID-19 patients.

FIG. 11. Graphs showing correlation between results for GPF of the COVID positive persons and the biomarkers D-dimer, ferritin, LDH, IL-6, CRP, and fibrinogen.

FIG. 12. A ROC curve (A) and Youden's index (B) to determine COVID-19 status with GPF level. The AUC for γ′ fibrinogen was 0.91 (95% CI: 0.85-0.98). Youden's Index showed a cut-point of 46.9 mg/dl between healthy controls (COVID-free subjects) and COVID-19 infected persons. The sensitivity at this cut-point was 0.81 and the specificity was 0.84.

FIG. 13. ROC curves to determine COVID-19 status with the six biomarkers: (A) The AUC for CRP was 0.51 (95% CI: 0.37-0.64); (B) The AUC for IL-6 was 0.63 (95% CI: 0.51-0.75); (C) The AUC for ferritin was 0.68 (95% CI: 0.56-0.80); (D) The AUC for LDH was 0.61 (95% CI: 0.49-0.72); (E) The AUC for D-dimers was 0.72 (95% CI: 0.62-0.81); (F) The AUC for total fibrinogen was 0.55 (95% CI: 0.41-0.70).

FIG. 14. A curve showing the relationship between COVID-19 severity and GPF level. COVID-19 severity was assessed by SpO₂ levels, with mild defined as SpO₂>95, moderate as 91≤SpO₂<95, and severe as SpO₂<91. There was a significant difference in mean γ′ level by group (p=0.0008), driven by the higher mean in the severe group.

FIG. 15. A ROC curve (A) and Youden's index (B) to determine COVID-19 severity with GFP. The AUC for γ′ fibrinogen was 0.77 (95% CI: 0.64-0.90). Youden's index showed a cut-point of 67.4 mg/dL between mild/moderate vs. severe disease. The sensitivity at this cut-point was 0.88 and the specificity was 0.62.

FIG. 16. Boxplots showing relationship between COVID-19 severity and the six other biomarkers: (A) The p value for CPR was 0.32; (B) The p value for IL-6 was 0.077; (C) The p value for ferritin was 0.20; (D) The p value for LDH was 0.0003; (E) The p value for D-dimers was 0.0007; (F) Total fibrinogen was normally distributed, and linear regression showed a p value of 0.68.

FIG. 17. ROC curves to determine COVID-19 severity with the six biomarkers AUC curves were used to compare mild/moderate vs. severe disease: (A) The AUC for CRP was 0.62 (95% CI: 0.47-0.77); (B) The AUC for IL-6 was 0.34 (95% CI: 0.21-0.46); (C) The AUC for ferritin was 0.56 (95% CI: 0.41-0.71); (D) The AUC for LDH was 0.82 (95% CI: 0.72-0.91); (E) The AUC for D-dimers was 0.73 (95% CI: 0.60-0.87); (F) The AUC for total fibrinogen was 0.56 (95% CI: 0.39-0.72).

### DESCRIPTION OF PREFERRED EMBODIMENTS

In order to provide a more precise understanding of the person matter of the invention, features of the invention will now be discussed with reference to the following preferred embodiment or embodiments.

### Experimental Study 1—Biomarkers of COVID-19

Primary objective: Evaluation of γ′ fibrinogen concentration levels in plasma samples of persons infected with COVID-19 ('COVID-19 patients') and healthy COVID-free persons ('COVID-free subjects').

Secondary objective: Evaluation of correlation of γ′ fibrinogen (GPF) levels with the CT value (by RT-PCR) and other inflammatory parameters including C-reactive protein (CRP), IL-6, ferritin, LDH and D-dimer.

### Ethics

This study was approved by the Institutional ethical committee (IEC., Micro therapeutics Laboratory, Chennai, India and Saveetha medical college, Chennai, India) on 27 May 2021. All invitees were provided with written information about the study and they were given the opportunity to ask questions. Participants gave written informed consent prior to completing the questionnaires. No compensation was provided to participants.

### Study Person Selection

This study was carried out in Micro therapeutics laboratory (Chennai, India)

Inclusion criteria:
1. COVID-19 patients meeting of all the following criteria were considered for enrolment in the study:

a) Voluntarily participating in the research study; fully understanding and being fully informed of the study.

b) having signed the Informed Consent Form (ICF); willingness and capability to complete all the study procedures.

c) Age 18-65 years (both inclusive) at the time of signing ICF.

d) Patients with laboratory confirmation of infection with SARS-COV-2 by positive RT-PCR.

e) Patients asymptomatic to critically ill.

f) Not participating in any other interventional drug clinical studies before completion of the present study.
2. Healthy COVID-19-free subjects meeting all of the following criteria will be considered for enrolment in the study:

a) Healthy COVID-19-free subjects had no known bleeding disorder, liver or kidney disease, cancer, or history of surgery or thrombotic event within the past 3 months.

b) Voluntarily participating in the clinical study; fully understanding and being fully informed of the study and having signed the (ICF); willingness and capability to complete all the study procedures.

c) With no previous history of viral infection and other diseases. Individuals with laboratory confirmation of infection with SARS-COV-2 by negative RT-PCR.

d) Age 18-65 years (both inclusive) at the time of signing ICF.

Exclusion criteria:
1. COVID-19 patients with history or significant presence of the following were excluded from participation/enrolment in the study trial:

a) Participation in this study will not be in the best interest of the person, or any other circumstances that prevent the person from participating in the study safely.

b) Pregnant or lactating women.

c) Having participated in any other interventional drug clinical study within 30 days prior to first dose of study drug.

d) Any disease or inflammatory conditions like rheumatoid arthritis, Crohn's disease.
2. Healthy person with history or significant presence of the following will be excluded from participation/enrolment in the study trial

a) Person who had participated in any other study within the 90 days of check-in.

b) History of difficulty in the accessibility of veins.

### Sample Collection

Initial screening and evaluation were performed with a small group of subjects with a sample size (N=73) who were diagnosed positive for COVID-19 by RT-PCR (COVID-19 patients), and (N=19) who were diagnosed negative for COVID-19 (no previous exposure to viral infection and other diseases, i.e. ‘COVID-free subjects’).

Blood sample was collected from the COVID-19 patients and healthy COVID-free subjects.

### Laboratory Testing

2 ml of whole blood was collected in a 3.2% sodium citrate tube and transported to the laboratory at ambient room temperature (15-25° C.).

Inflammatory test profile for C-reactive protein, ferritin, IL-6, LDH, D-dimer, and total fibrinogen was analysed by using validated analytical methods.

Ct values (RT-PCR) was obtained from the Micro Therapeutics Company for all COVID-19 positive patients including controls (COVID-free subjects).

γ′ fibrinogen (GPF) was analysed by using ELISA quantification Kit (Gamma Coeur ELISA Kit-GCEK001).

### Statistical Analysis

Analysis of the Endpoint(s) was performed using Mann-Whitney U test using the GraphPad Prism6.

### Results

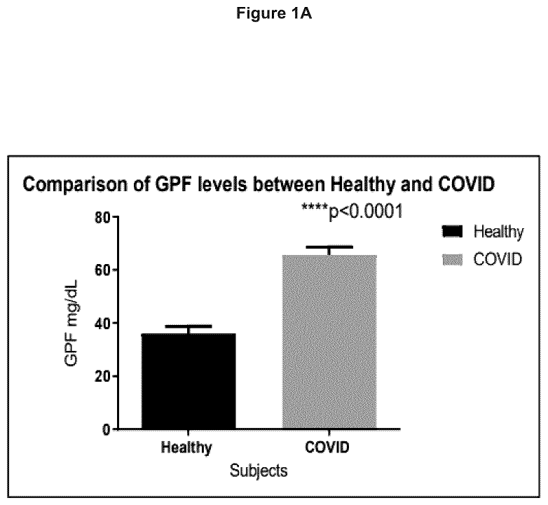

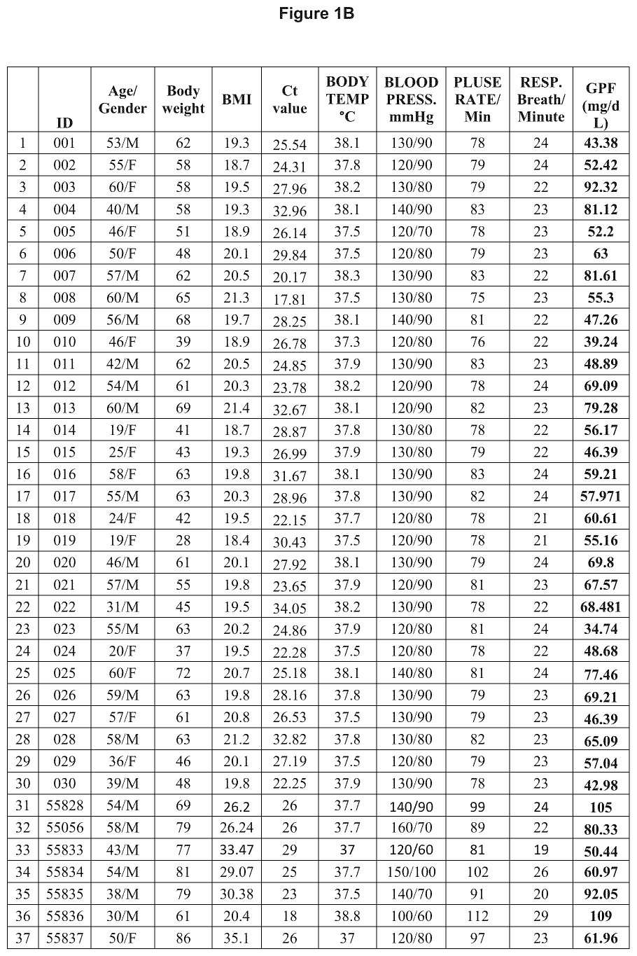

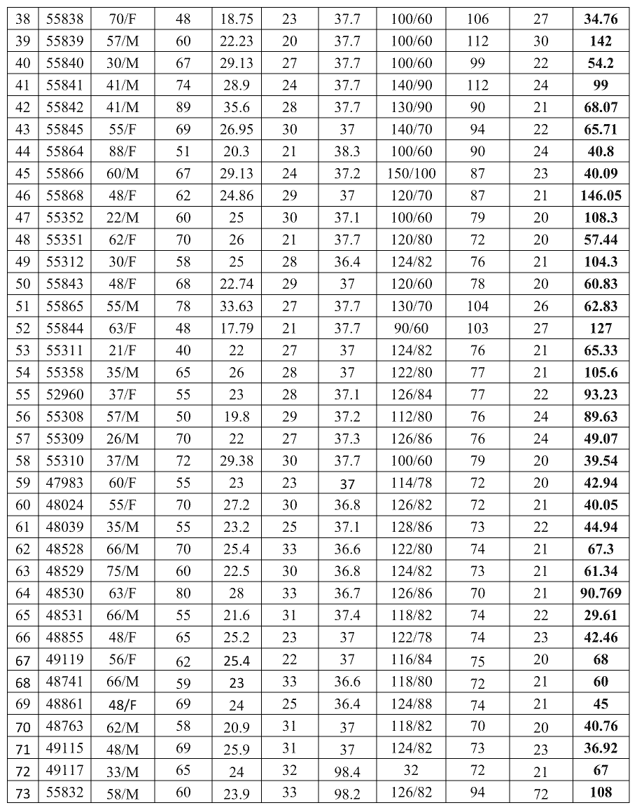

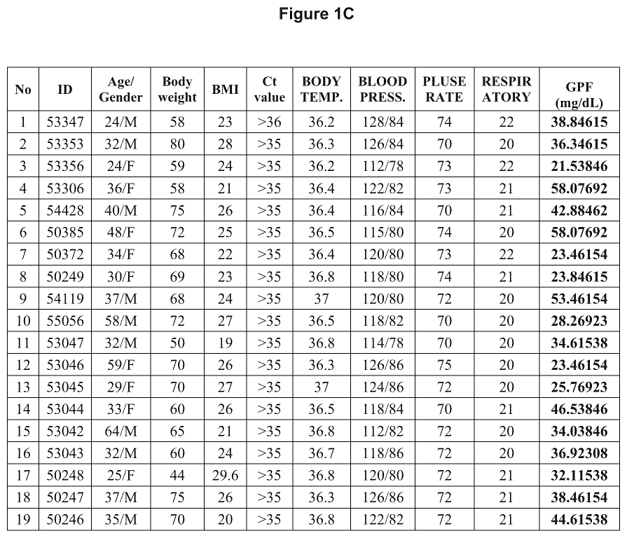

In the study, the level of γ′ fibrinogen (GPF) was compared in plasma samples of 19 healthy COVID-19-free subjects and 73 COVID-19 patients. The concentration of γ′ fibrinogen was assessed using (GammaCoeur ELISA Kit-GCEK001). C-reactive protein, IL-6, LDH, ferritin, D-dimer and CT value was analysed in both COVID-19-free subjects and COVID-19 patients by using validated analytical methods. In our study we found that γ′ fibrinogen is elevated in COVID-19 patients in comparison to COVID-19-free subjects (controls) (FIG. 1) with a significance of (*** p<0.0001).

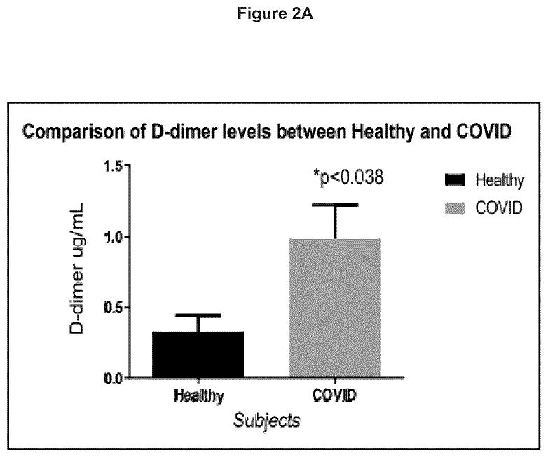

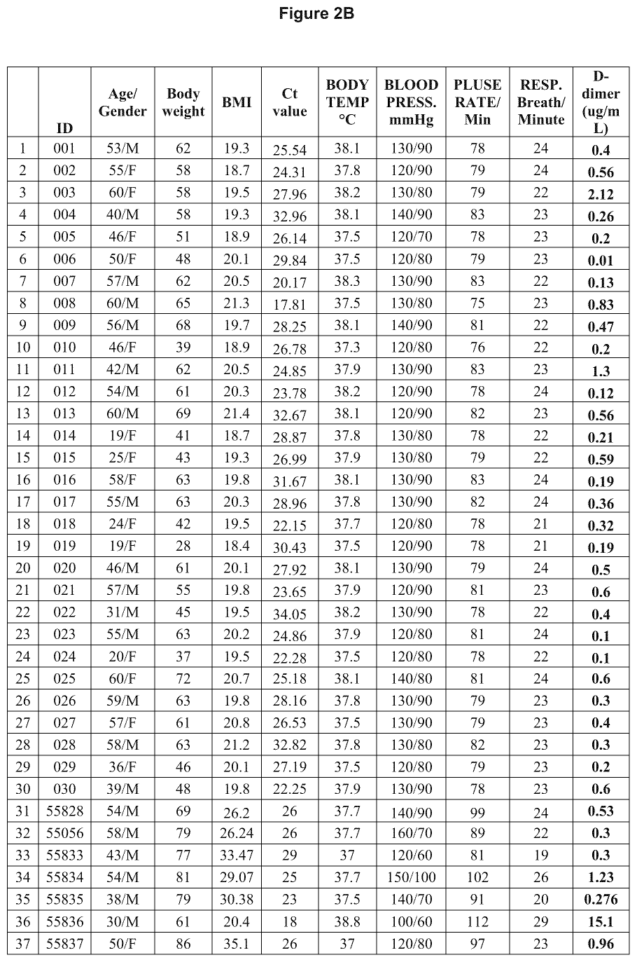

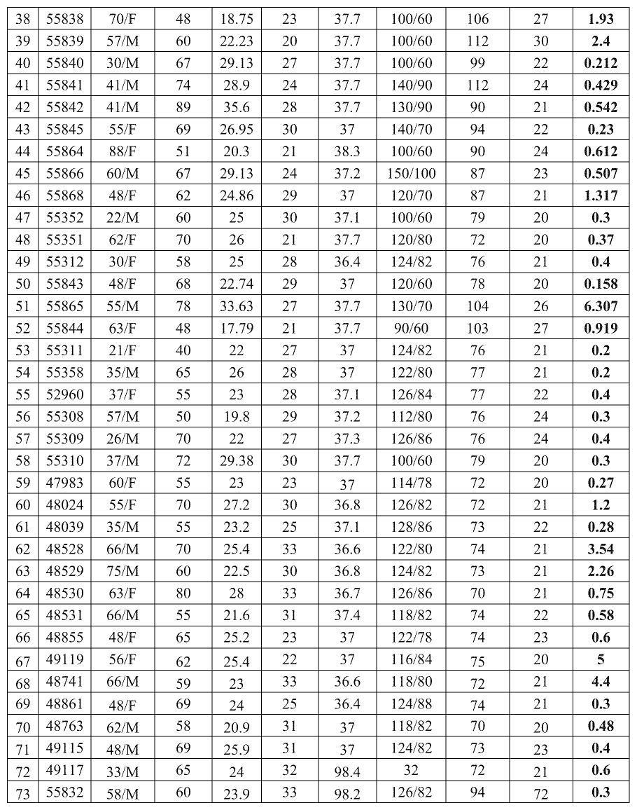

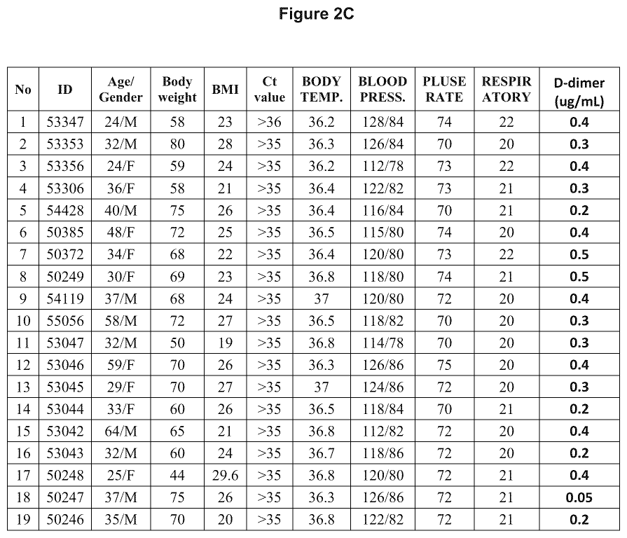

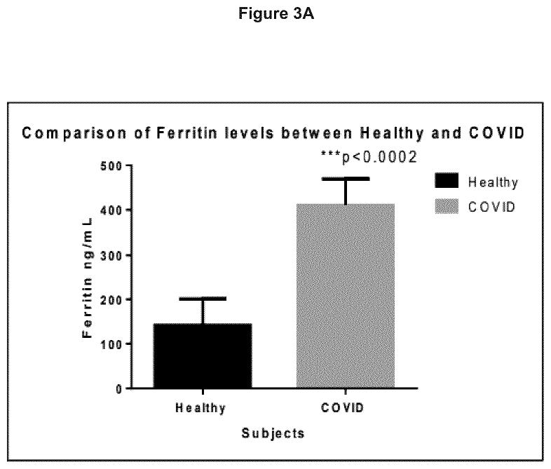

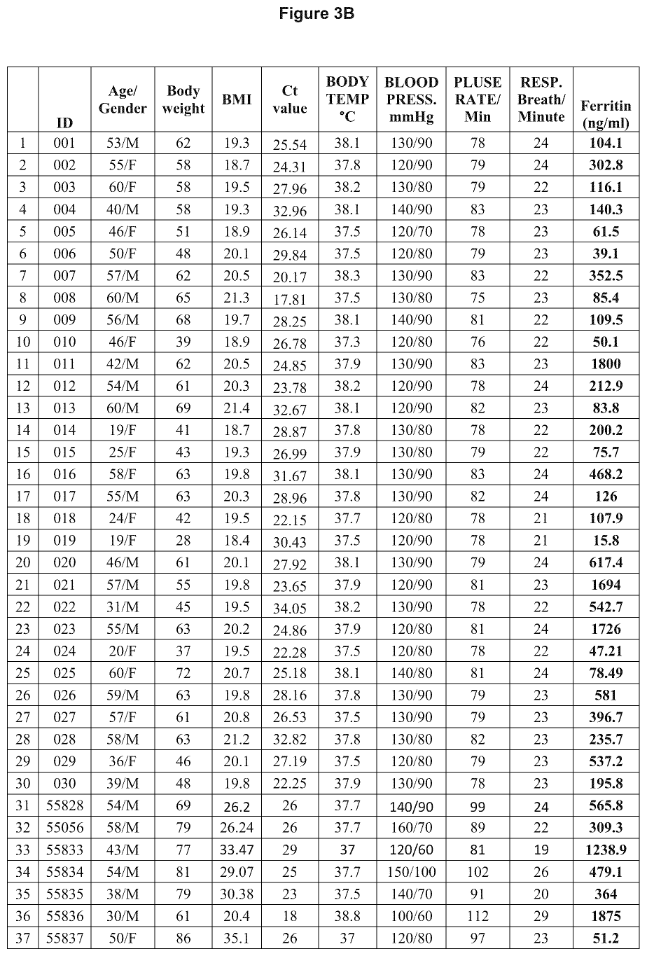

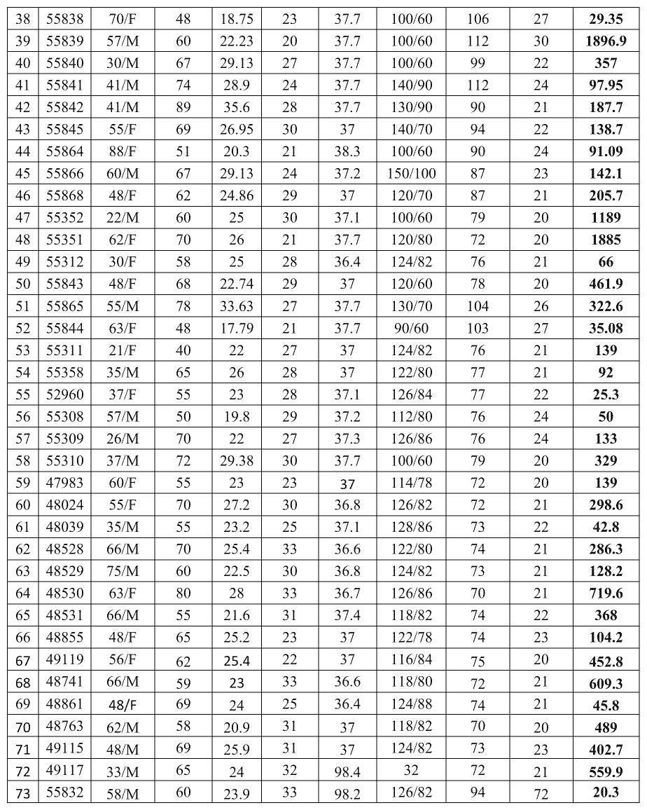

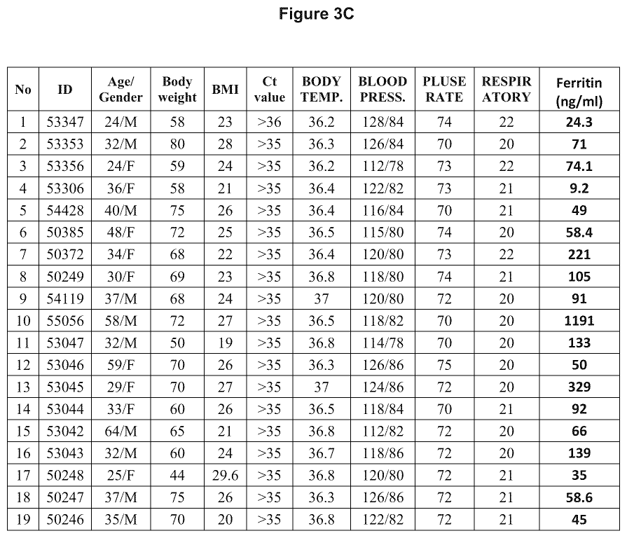

In addition, it is also found that the level of D-dimer and ferritin in COVID-19patients in comparison to COVID-19-free subjects with a significance of ** p<0.038 (FIG. 2) and *** p<0.0002 (FIG. 3), respectively.

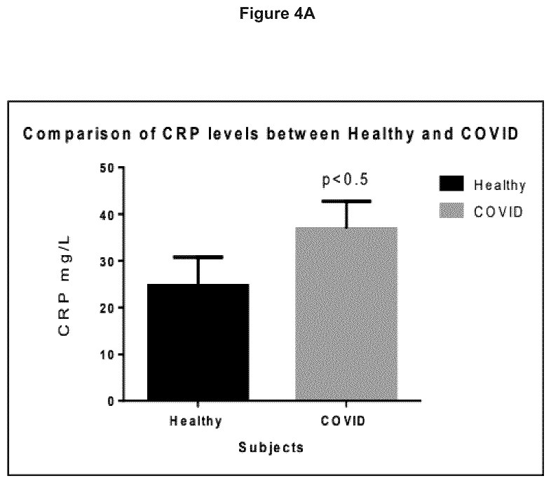

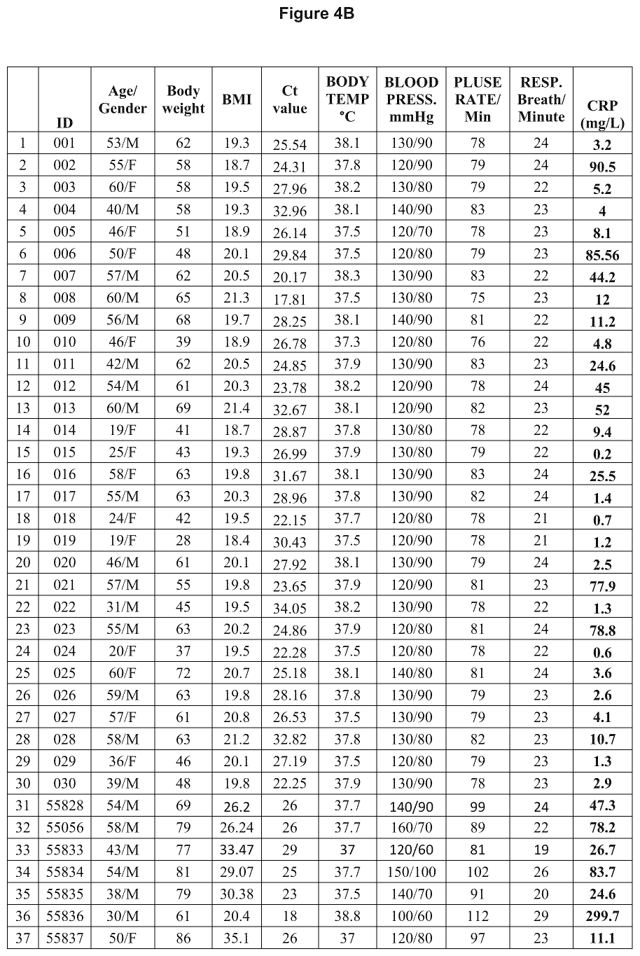

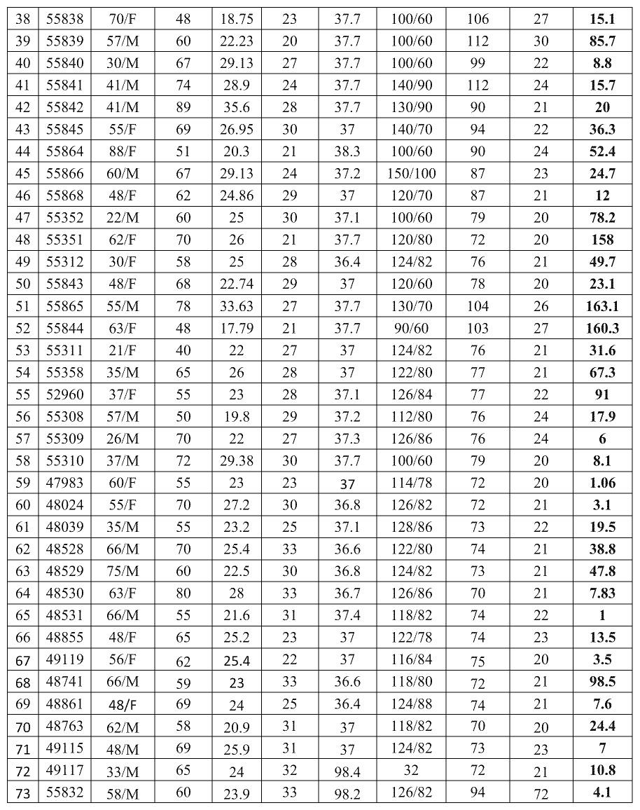

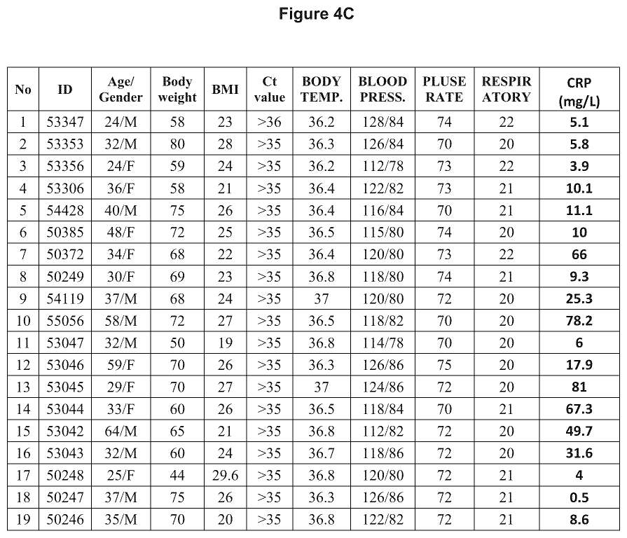

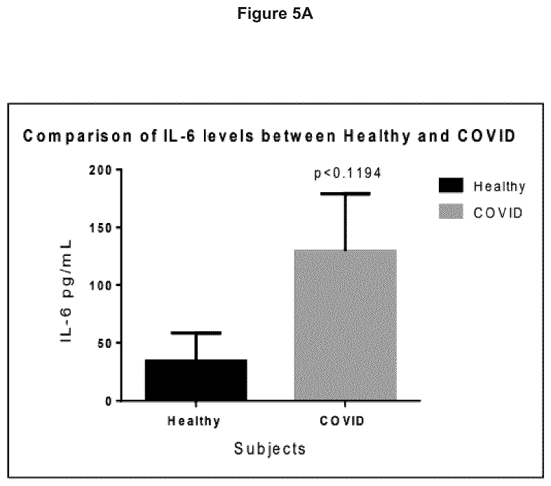

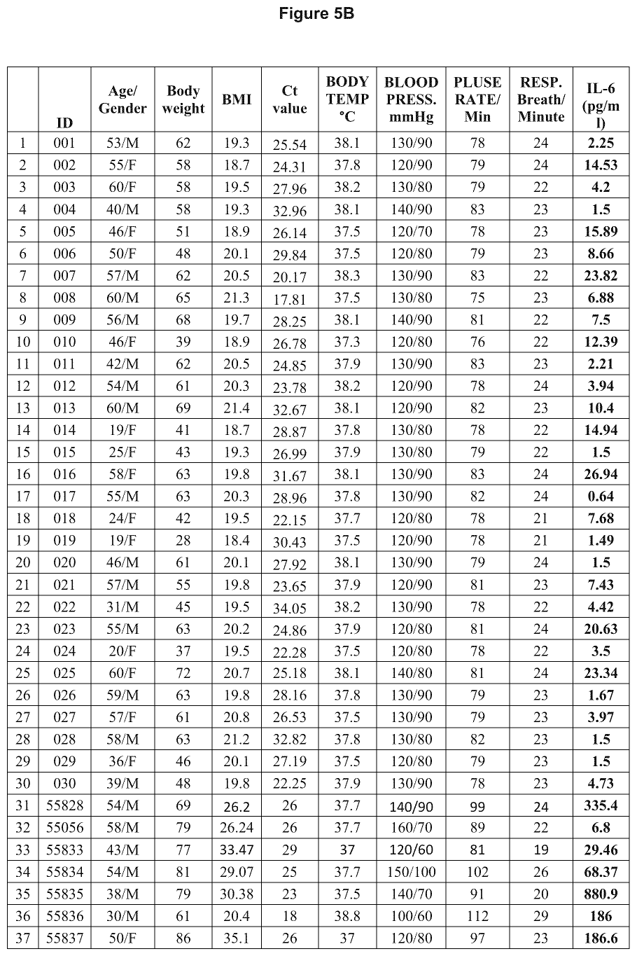

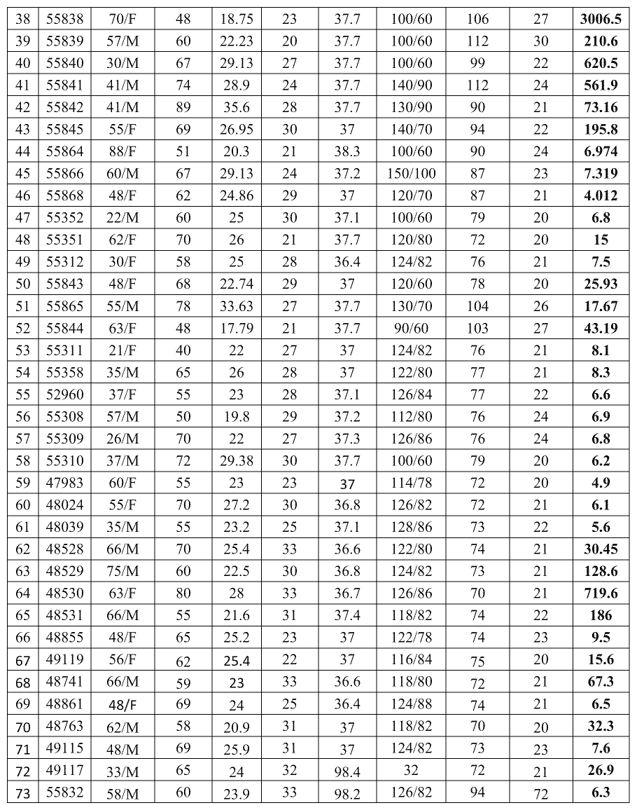

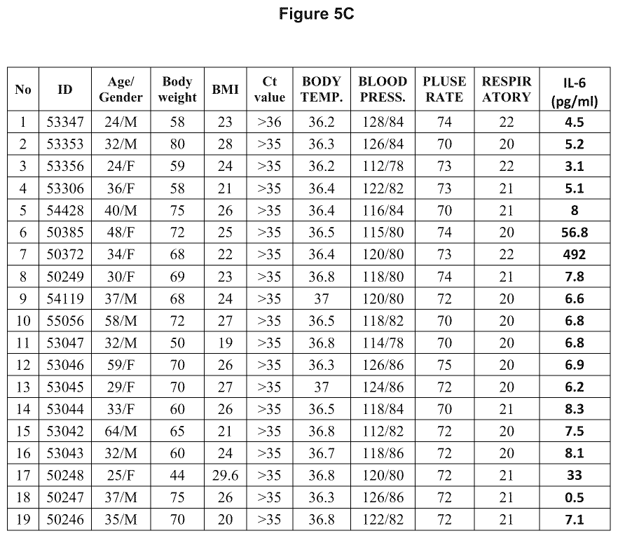

Other parameters like CRP and IL-6, the levels are increased in COVID-19 patients in comparison to COVID-free subjects, however there is no significance with p values with p<0.5 (FIG. 4), and p<0.1194 (FIG. 5), respectively.

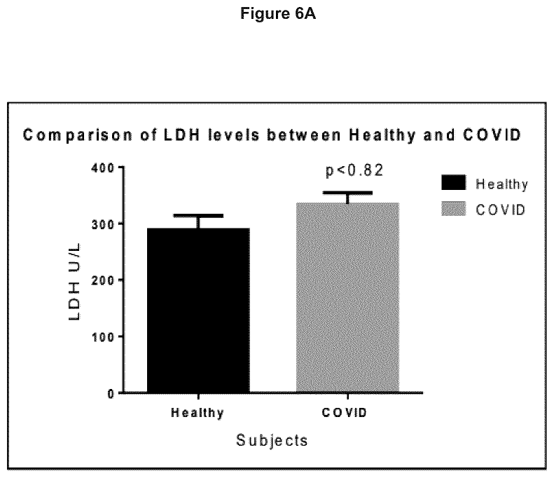

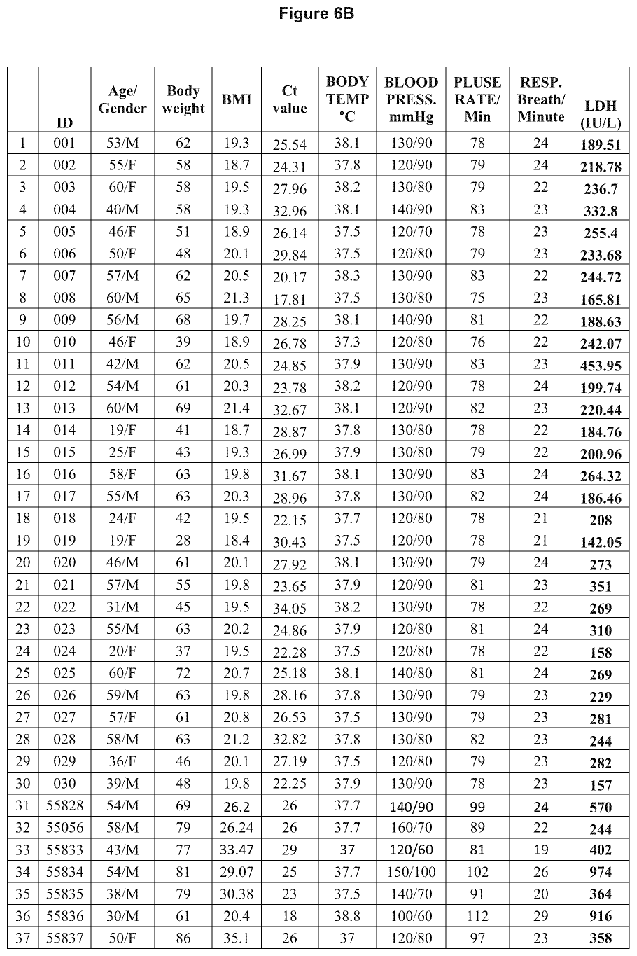

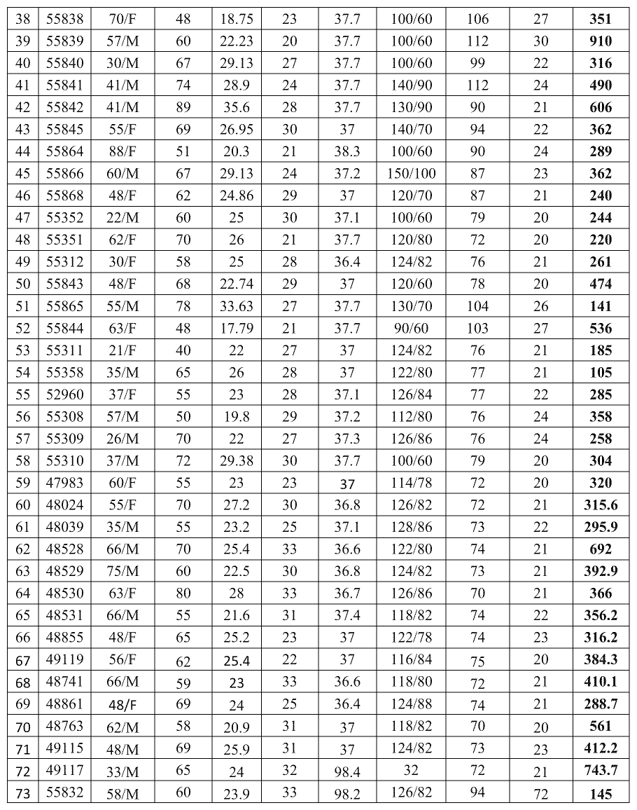

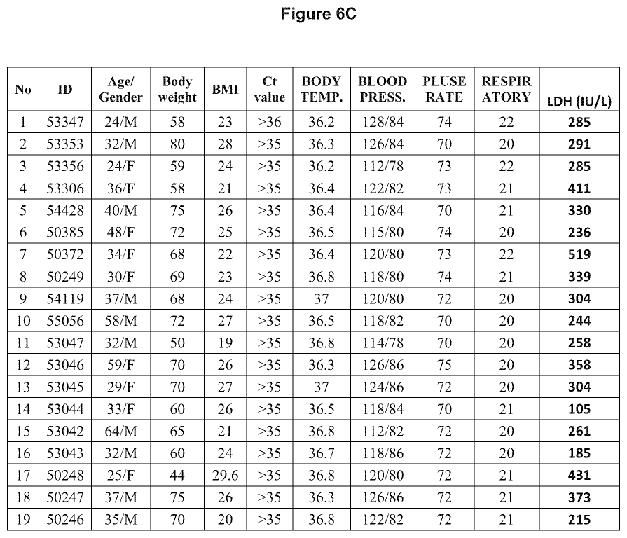

However, we also observed equivocal results with LDH in COVID-19 patients in comparison to COVID-19-free subjects with p values of p<0.82 (FIG. 6).

However, it was observed that there is no correlation between the cT value and γ′ fibrinogen.

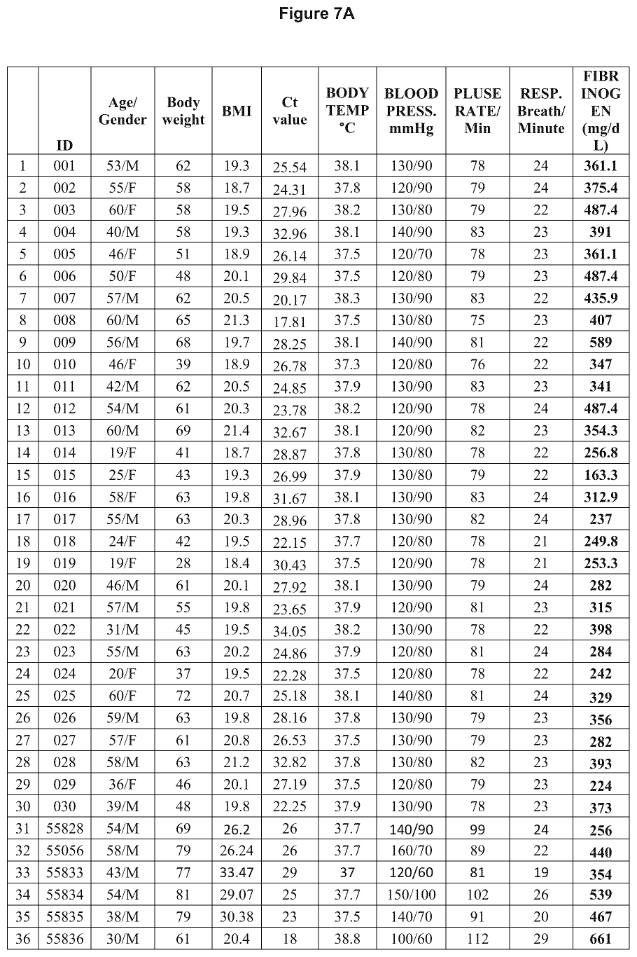

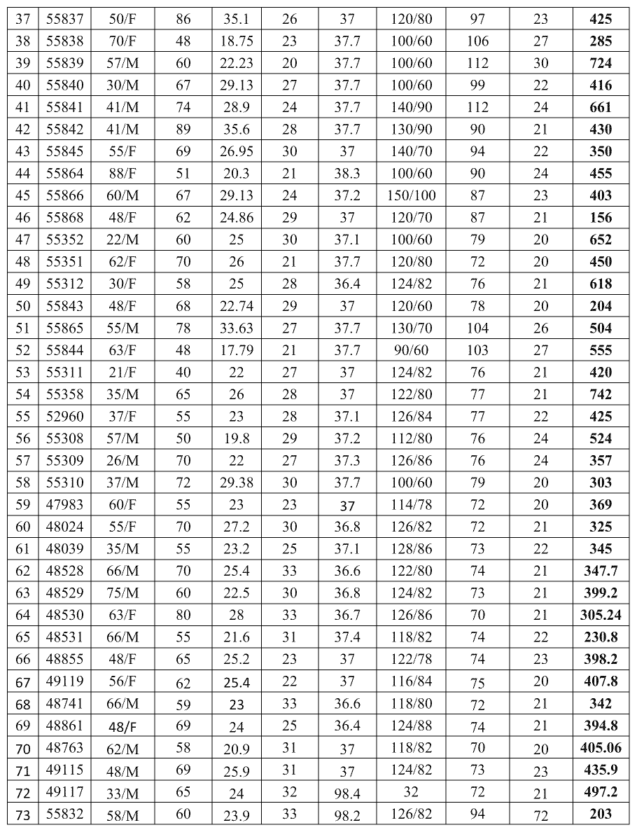

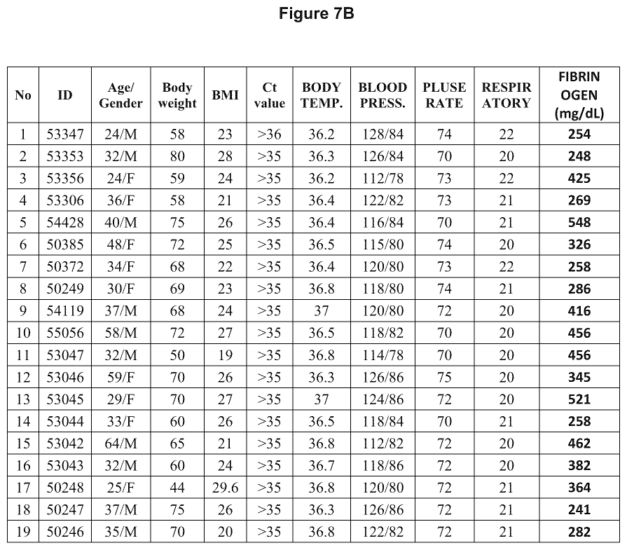

Data for total fibrinogen in COVID-19 patients in comparison to COVID-19-free subjects were also calculated and provided in FIG. 7.

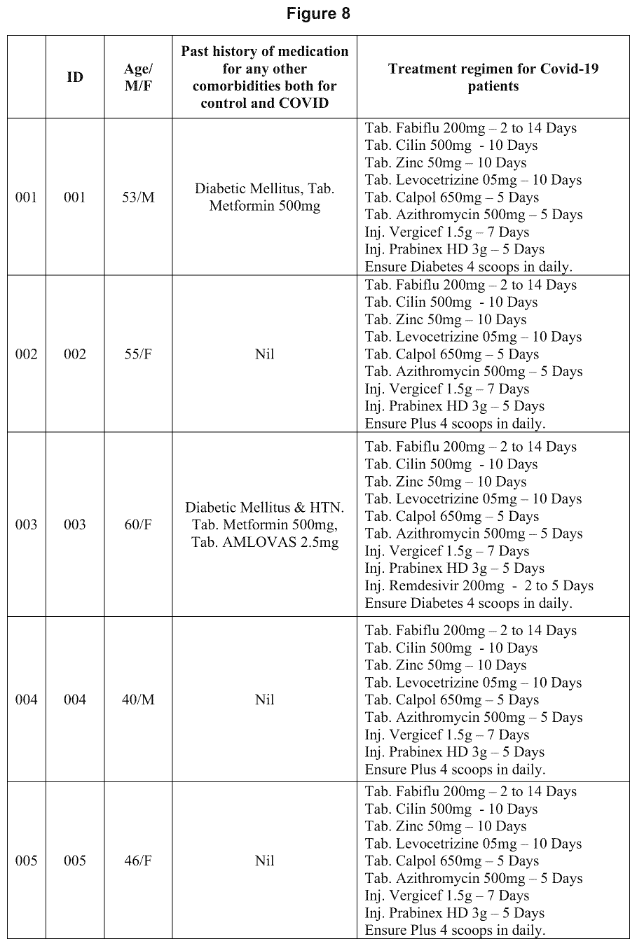

The treatment regime for treating COVID-19 in the COVID-19 patients, in addition to any comorbidities and associated treatment regime in the same persons are provided in FIG. 8. It is further noted that none of the COVID-19 patients required ventilation or died as a result of their COVID-19 infection during the study.

Therefore, this finding identifies an association between parameters comprising D-dimer, ferritin, CRP, and IL-6 levels, and γ′ fibrinogen with COVID-19 disease severity.

### Further Analysis

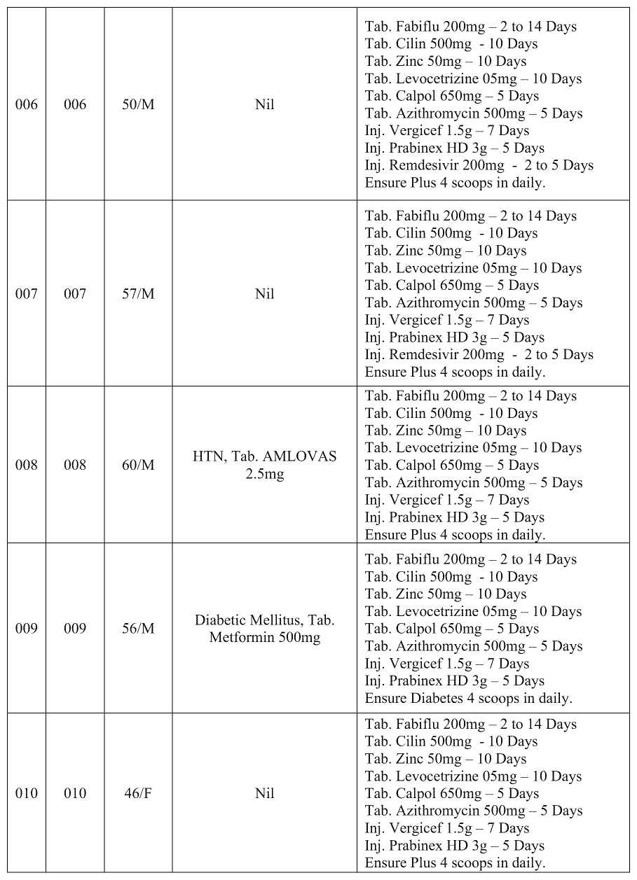

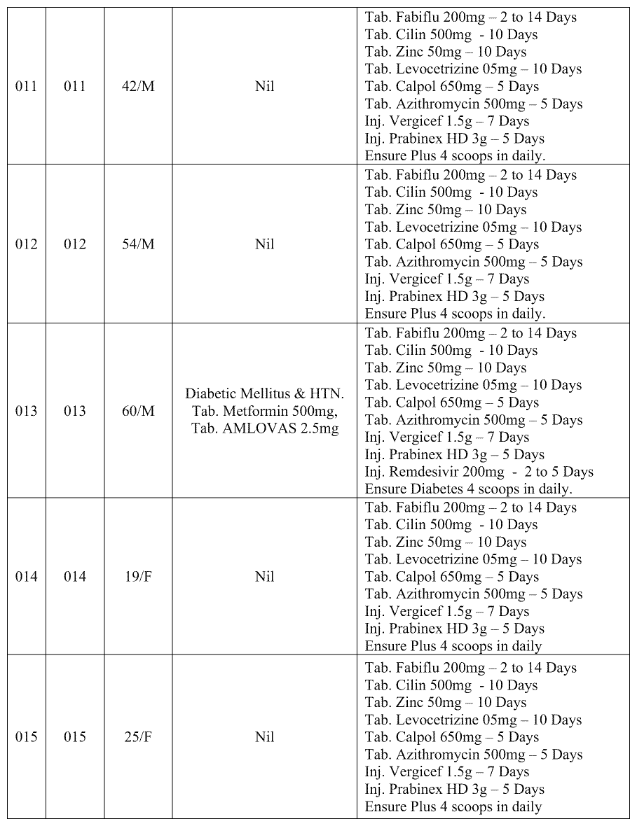

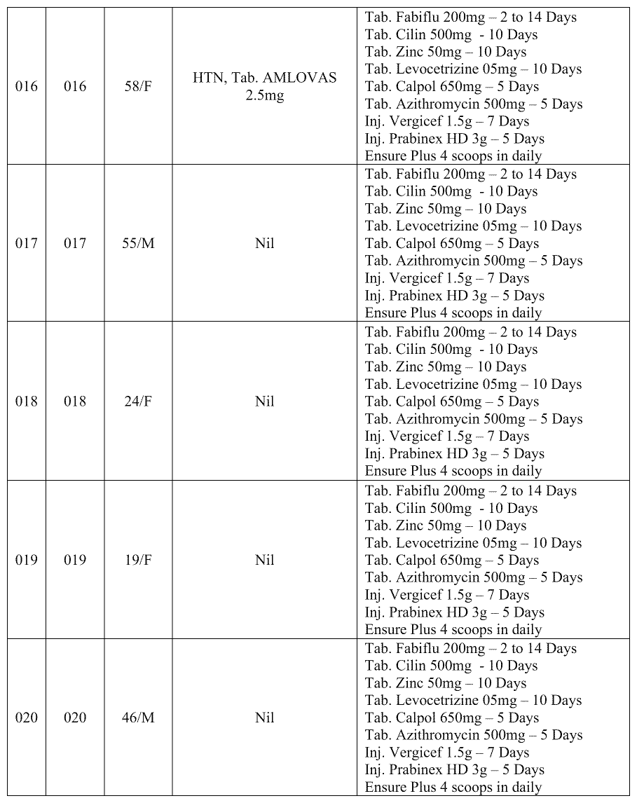

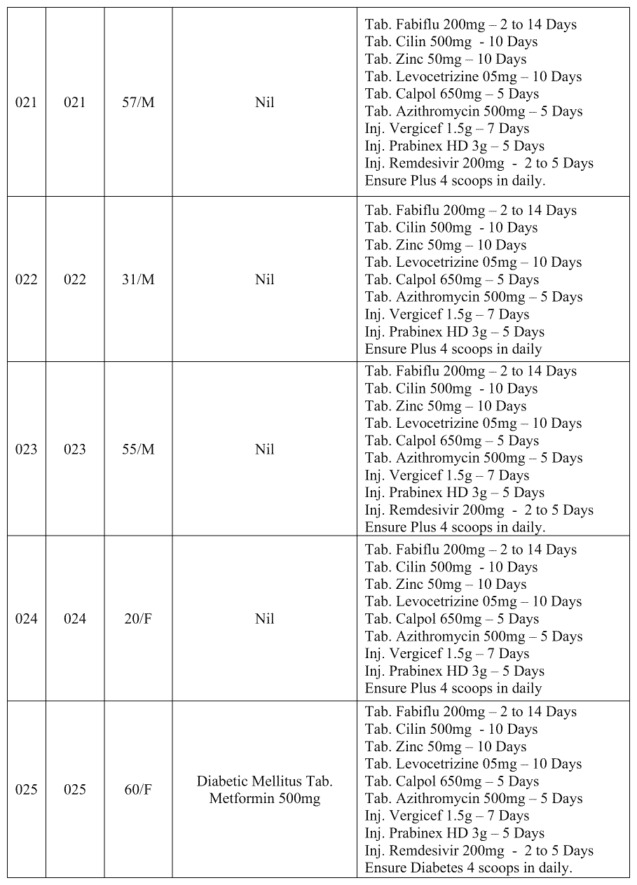

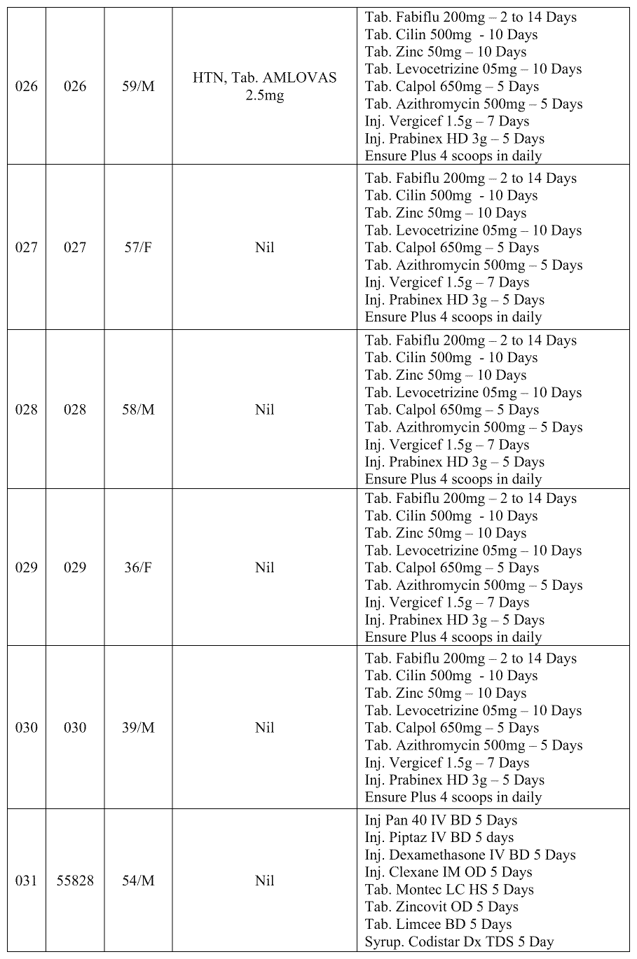

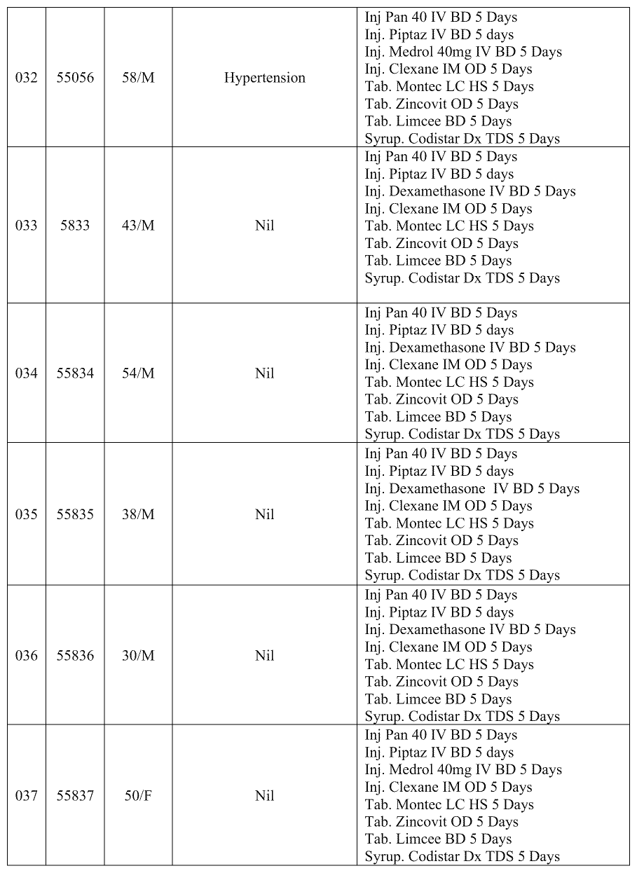

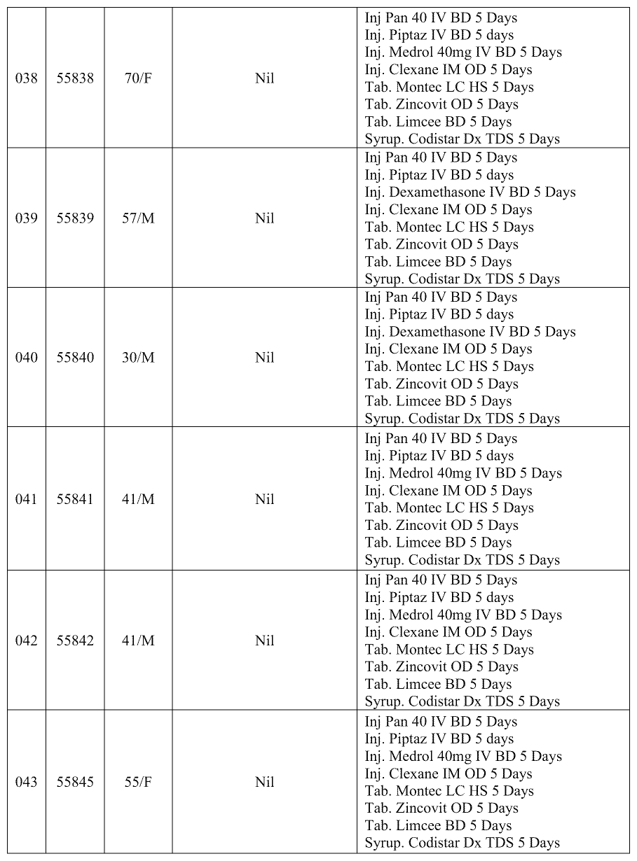

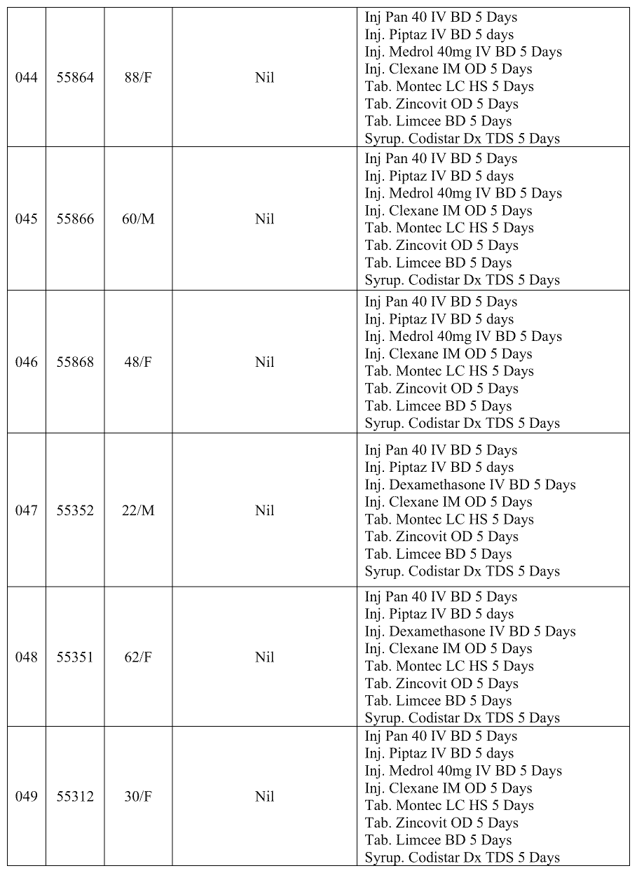

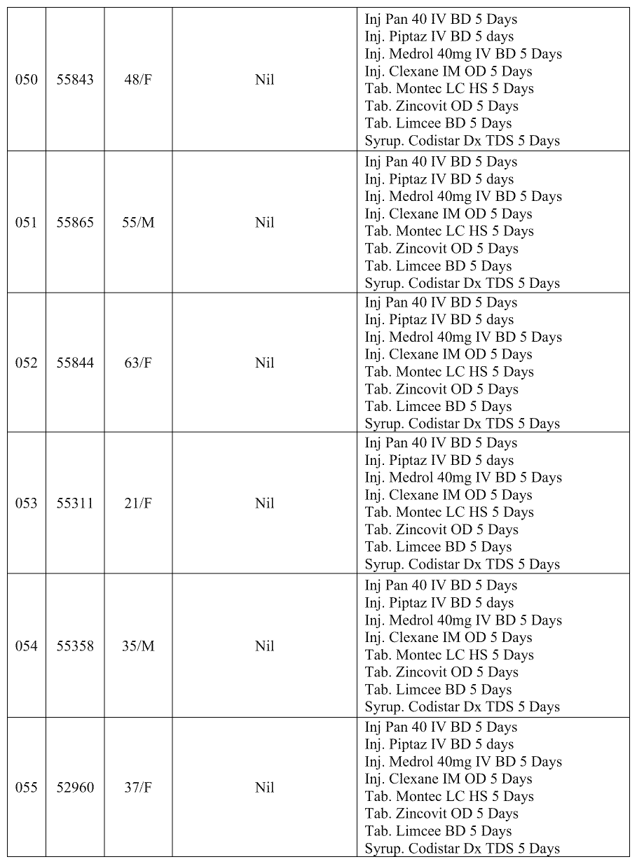

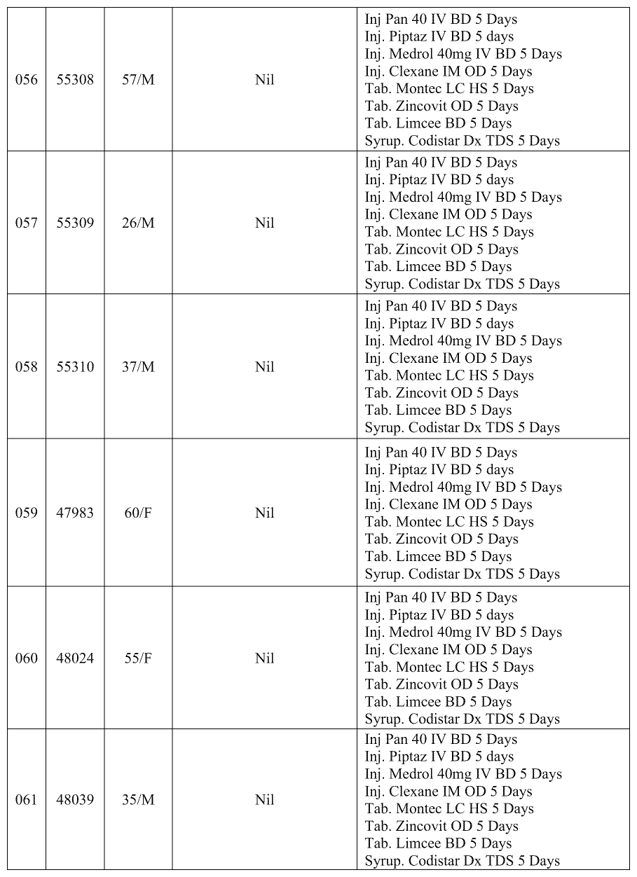

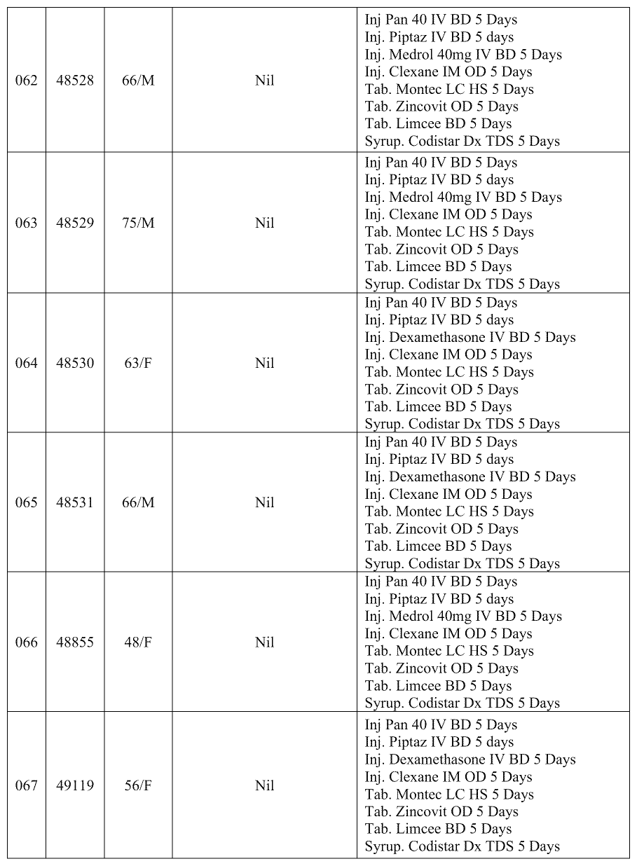

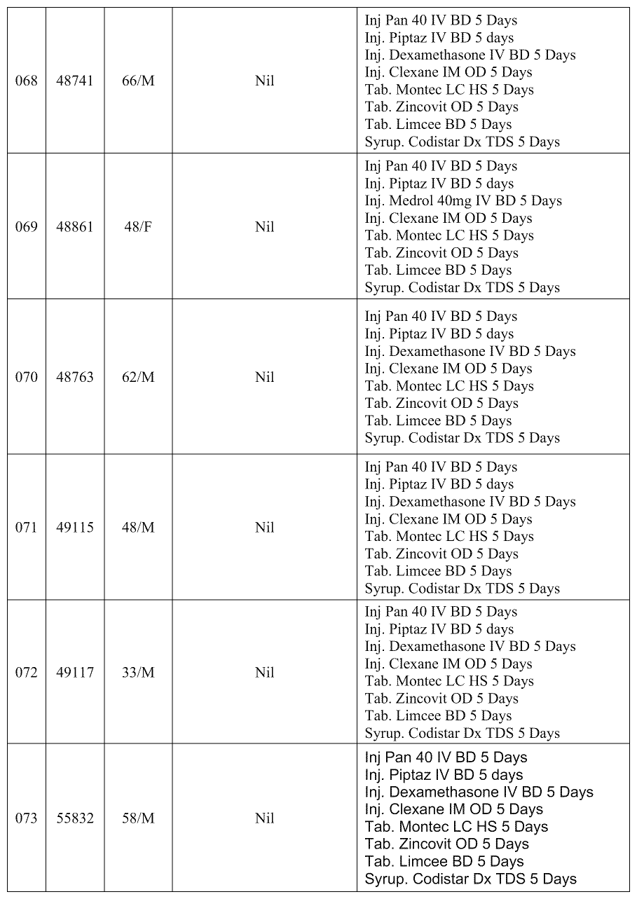

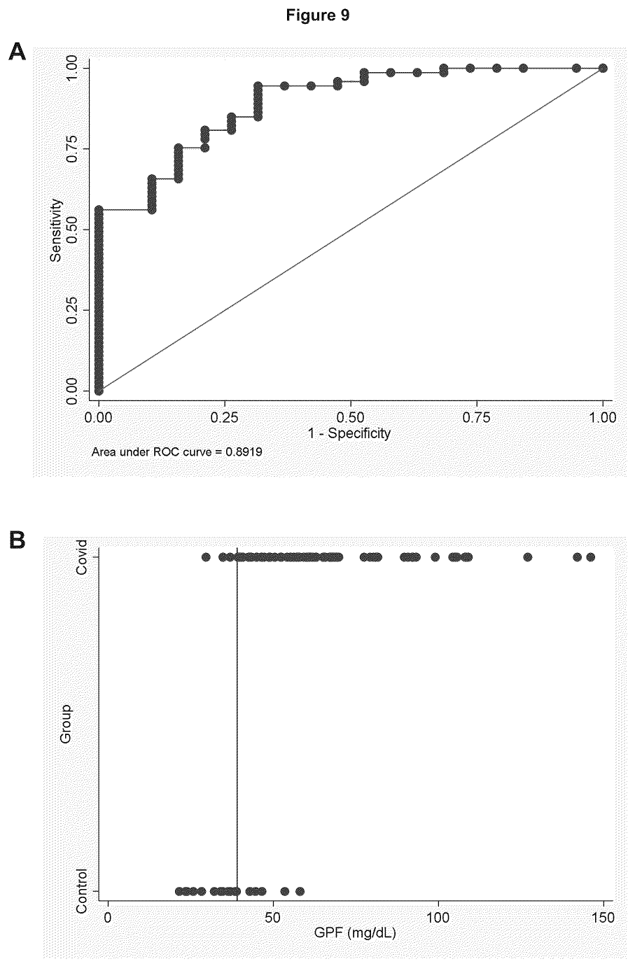

GPF data obtained in the study was used to generate a ROC curve to discriminate by COVID-19 status, and which was found to be beneficial producing an AUC of 0.89 (95% CI: 0.82-0.97) as shown in FIG. 9A.

Youden's index was then employed to generate a cut-point that maximizes both sensitivity and specificity. It identified 39.040375 as a cut-point, with 95% sensitivity and 68% specificity as shown in FIG. 9B.

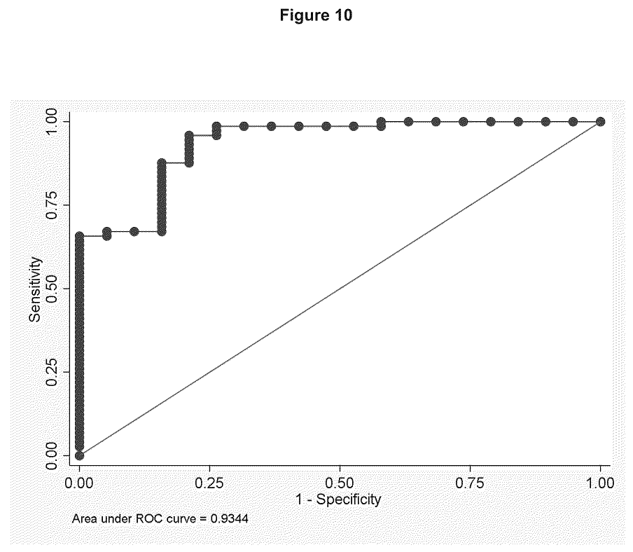

The ROC curve in FIG. 10 shows a logistic regression using GPF, D-dimer, and ferritin that was then calculated. While caution should be exhibited analysing the statistical significance of the results due to the relatively small number of COVID-free subjects, the combination of the three biomarkers appeared to be useful for discriminating between COVID-19 patients in comparison to COVID-19-free subjects with an AUC=0.93 (95% CI: 0.87-0.99).

The correlation between GPF and the biomarkers D-dimer, ferritin, LDH, IL-6, CRP, and fibrinogen was analysed for the COVID-19 patients.

The results are shown in the graphs in FIG. 11.

A p-value of 0.0083 was used to correct for looking at six comparisons (0.05/6=0.0083). We concluded from the data that CRP and fibrinogen are correlated. Note that the correlation coefficient is the top number, and the p-value is the lower number as shown in Table 1.

TABLE 1

| | gpfmgdl |
|---|---|
| gpfmgdl | 1.0000 |
| ddimerugml | 0.2153 |
| | 0.0673 |
| ferritinngml | 0.1436 |
| | 0.2254 |
| ldhiul | 0.2459 |
| | 0.0360 |
| i16pgml | −0.0242 |
| | 0.8392 |
| crpmgl | 0.3580* |
| | 0.0019 |
| fibrinogen~l | 0.4115* |
| | 0.0003 |

### Experimental Study 2—Prognosis of COVID-19 Severity

Primary objective: Evaluation of levels of γ′ fibrinogen and association with SpO₂ levels in COVID-19 patients compared to healthy COVID-free subjects.

Ethics and Study person selection as described above for Experimental Study 1.

### Laboratory Testing

Persons comprises COVID-19 patients (N=103) who were diagnosed positive for COVID-19 by RT-PCR, and (N=19) who were diagnosed negative for COVID-19 (no previous exposure to viral infection and other diseases, i.e. ‘COVID-free subjects’).

Blood sample was collected from the COVID-19 patients and healthy COVID-free subjects.

2 ml of whole blood was collected in a 3.2% sodium citrate tube and transported to the laboratory at ambient room temperature (15-25° C.).

An inflammatory test profile for total fibrinogen, D-dimers, IL-6, ferritin, LDH, and CRP was measured using standardized analytical methods.

Ct values (RT-PCR) were obtained from the Micro Therapeutics Company and Saveetha Hospital for all COVID-19 patients and COVID-19-free subjects (controls).

γ′ fibrinogen was analyzed using a commercial ELISA (Gamma Coeur ELISA Kit-GCEK001, Zeus Scientific).

### Statistical Analysis

The primary outcome was to assess for differences in γ′ fibrinogen levels among patients with mild (SpO2≥95), moderate (SpO2<95 and ≥91), and severe (SpO2<91) COVID-19 using a linear regression. We also examined the relationship between COVID-19 severity and the other biomarkers using Kruskal-Wallis test for skewed variables (IL-6, LDH, D-dimers, ferritin, and CRP) and linear regression for normally-distributed variables (total fibrinogen).

ROC curves were used to determine the ability of γ′ fibrinogen to differentiate between COVID-19 patients and COVID-free subjects, as well as between patients with severe COVID-19 versus mild-to-moderate cases. Area under the curve (AUC) and 95% confidence intervals (CI) were calculated. For each of these curves, Youden's index was calculated to determine the γ′ fibrinogen cut-point that maximizes both sensitivity and specificity. Sensitivity and specificity were then calculated at the cut-point. ROC curves, AUC, and 95% CI were also generated for the other biomarkers to explore their ability to discriminate between groups.

Within COVID-19 patients, correlation between γ′ fibrinogen and the other biomarkers was calculated using Spearman's rho due to the skewed distribution of most of the biomarkers. The relationship between BMI and γ′ fibrinogen was assessed using linear regression.

### Results

Biomarker Comparison between COVID-19 Patients and Healthy COVID-free Subjects

103 COVID-19 patients at Saveetha Medical College and Hospital, Chennai, India were identified by a positive RT-PCR test for Sars-COV-2 between Jun. 14, 2021 and Feb. 21, 2022. 19 healthy control volunteers (COVID-free subjects) were confirmed by a negative RT-PCR test. The characteristics of the participants are shown in Table 2.

TABLE 2
Patient Characteristics

| | Cases | Controls |
|---|---|---|
| Variable | (N = 103) | (N = 19) |
| Age-Mean (SD) | 49.2 (14.05) | 37.3 (11.74) |
| Gender-N (%) | | |
| Male | 59 (57.3) | 10 (52.6) |
| Female | 44 (42.7) | 9 (47.4) |
| BMI-Mean (SD) | 22.2 (3.87) | 24.3 (2.85) |
| Body temperature-Mean (SD) | 37.6 (0.51) | 36.6 (0.26) |
| SpO₂-Median (Q1, Q3) | 93 (92, 94) | 98 (96, 99) |
| Pulse rate-Median (Q1, Q3) | 79 (75, 83) | 72 (70, 73) |
| Respiratory rate-Mean (SD) | 22.7 (1.78) | 20.7 (0.75) |
| Systolic BP-Mean (SD) | 123.7 (10.87) | 119.7 (4.81) |
| Diastolic BP-Mean (SD) | 80.5 (9.52) | 82.3 (2.69) |

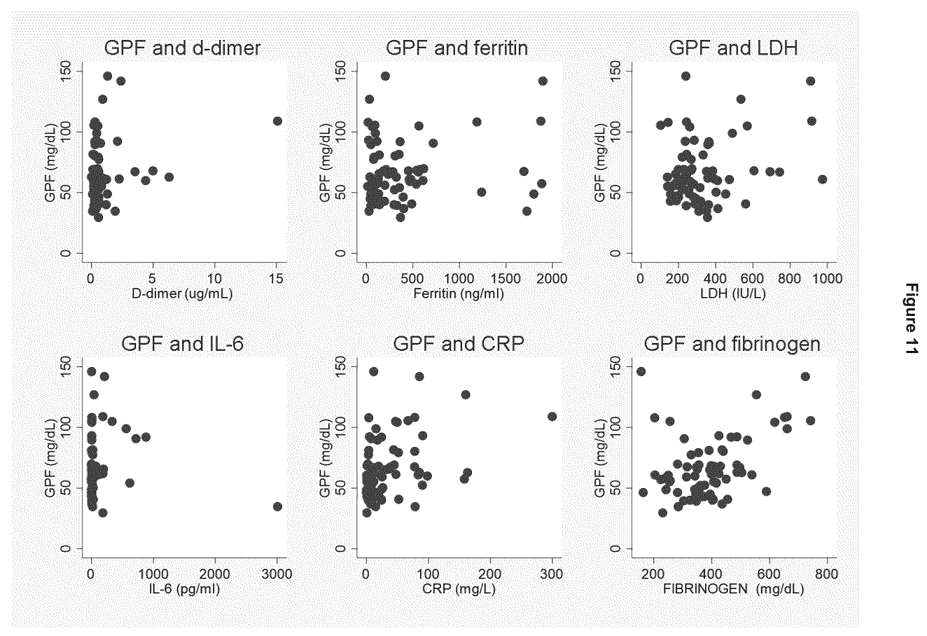

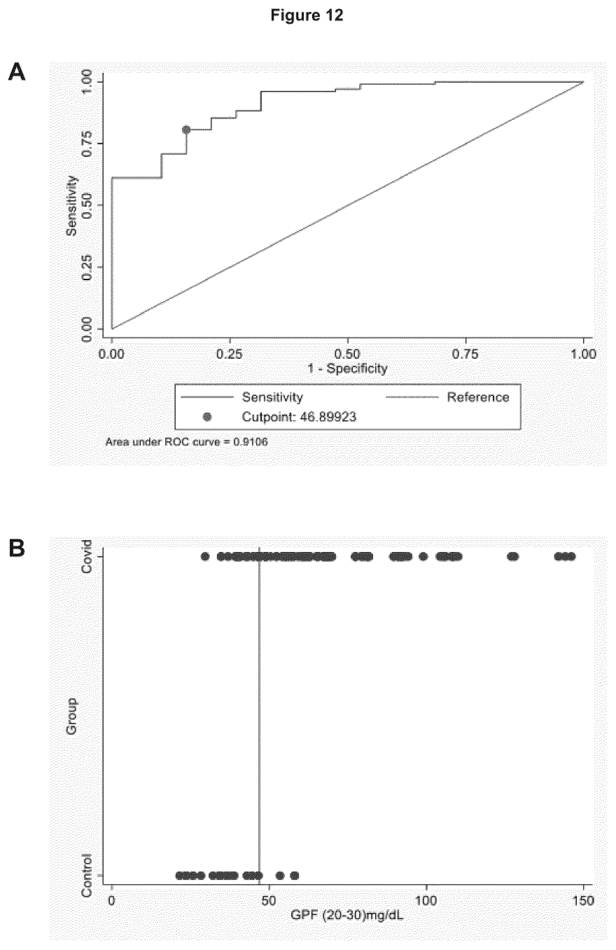

The levels of γ′ fibrinogen, CRP, IL-6, ferritin, LDH, D-dimers, and total fibrinogen in plasma samples of 103 COVID-19 patients were compared to 19 healthy COVID-free subjects. γ′ fibrinogen was significantly elevated in COVID-19 patients compared to COVID-19-free subjects (p<0.0001). The median level of γ′ fibrinogen in the COVID-19-free subjects was 36.4 mg/dl compared to 65.1 mg/dL in COVID-19 patients. The median for the COVID-19-free subjects was slightly higher than historical controls (29.3 mg/dL) in 10,601 patients in the Atherosclerosis Risk In Communities study (Appiah 2015). FIG. 12A shows a ROC curve for the association between γ′ fibrinogen levels and COVID-19 status. The AUC for γ′ fibrinogen was 0.91 (95% CI: 0.85-0.98). Youden's index (FIG. 12B) showed a cut-point of 46.9 mg/dL to best distinguish between healthy COVID-19-free subjects and COVID-19 patients. The sensitivity at this cut-point was 0.81 and the specificity was 0.84.

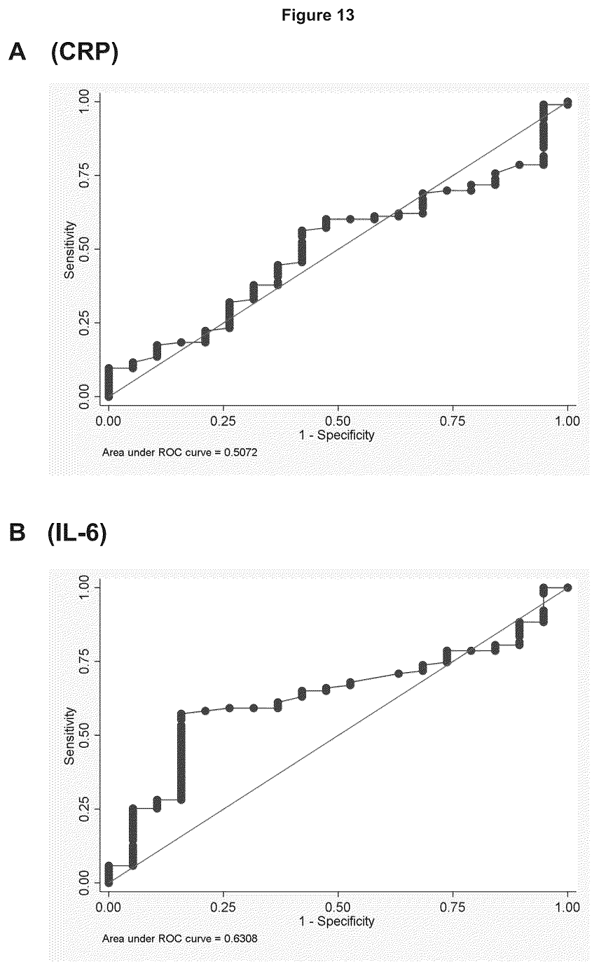

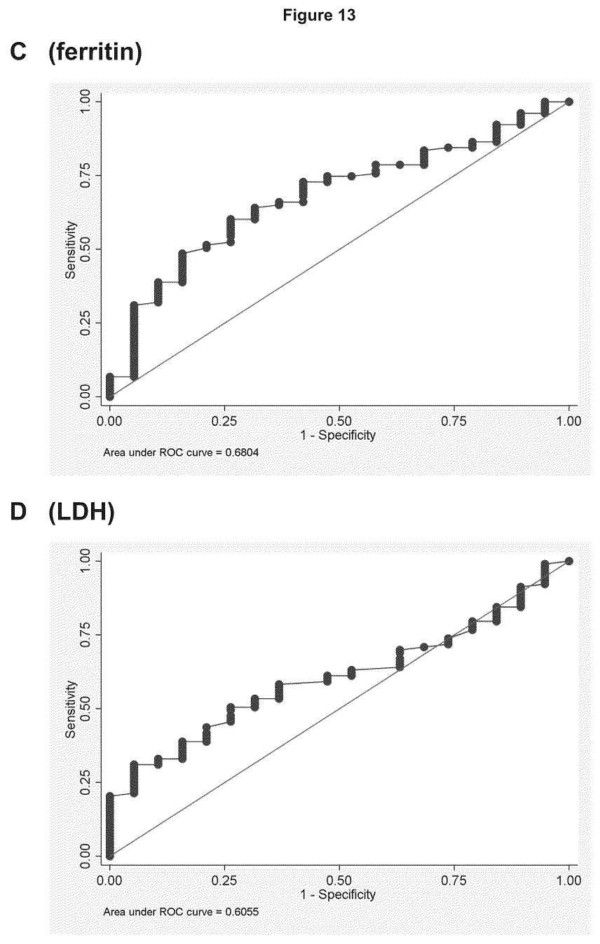

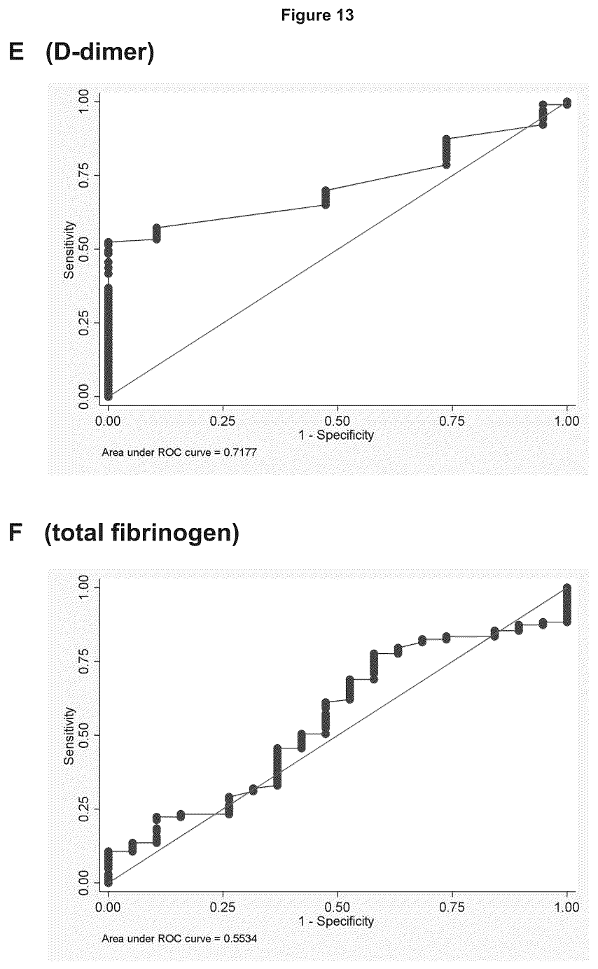

In contrast, CRP levels were not significantly different between COVID-19 patients and COVID-19-free subjects (p<0.8), and ROC curves for CRP showed an AUC of 0.51 (95% CI: 0.37-0.64) (FIG. 2A). IL-6 levels were significantly elevated in COVID-19 patients compared to COVID-19-free subjects (p<0.02) (FIG. 13B) as were ferritin levels (p<0.008) (FIG. 13C), LDH levels (p<0.08) (FIG. 13D), and D-dimers (p<0.0019) (FIG. 13E). ROC curves for IL-6 showed an AUC of 0.63 (95% CI: 0.51-0.75) (FIG. 13B), those for ferritin showed an AUC of 0.68 (95% CI: 0.56-0.80) (FIG. 13C), those for LDH showed an AUC of 0.61 (95% CI: 0.49-0.72) (FIG. 13D), and those for D-dimers showed an AUC of 0.72 (95% CI: 0.62-0.81) (FIG. 13E). Total fibrinogen levels were not significantly different between COVID-19 patients and COVID-19-free subjects (p<0.4) (FIG. 13F), with ROC curves showing an AUC of 0.55 (95% CI: 0.41-0.70) (FIG. 13F), suggesting that total fibrinogen levels were not simply a surrogate for γ′ fibrinogen levels.

Biomarker Comparisons with γ′ Fibrinogen Levels

The correlation between γ′ fibrinogen levels and CRP, IL-6, ferritin, LDH, D-dimers, and total fibrinogen were calculated within COVID-19 patients. Spearman's rho analysis showed that γ′ fibrinogen levels were not significantly correlated with IL-6 (p=0.28), ferritin (p=0.72), D-dimers (p=0.069), or fibrinogen (p=0.0509). In contrast, γ′ fibrinogen levels were significantly associated with CRP (p=0.019; rho=0.23) and LDH (p=0.0064; rho=0.27), although the rho values did not suggest a robust association. There was also no significant association between γ′ fibrinogen and BMI (p=0.84).

Biomarker Comparison with COVID-19 Severity

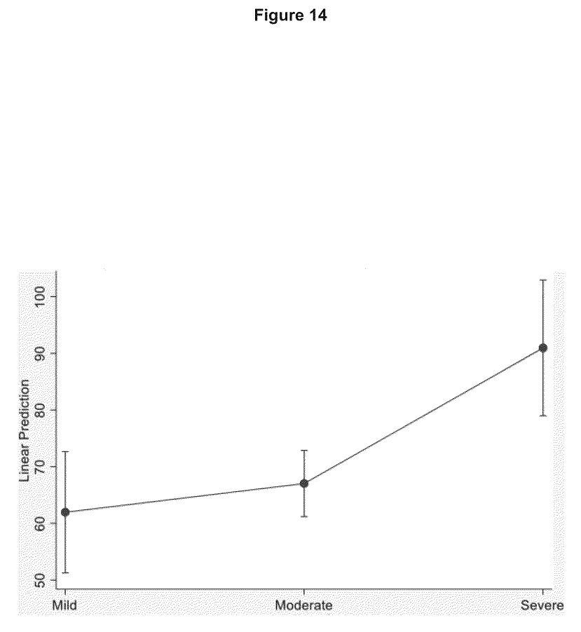

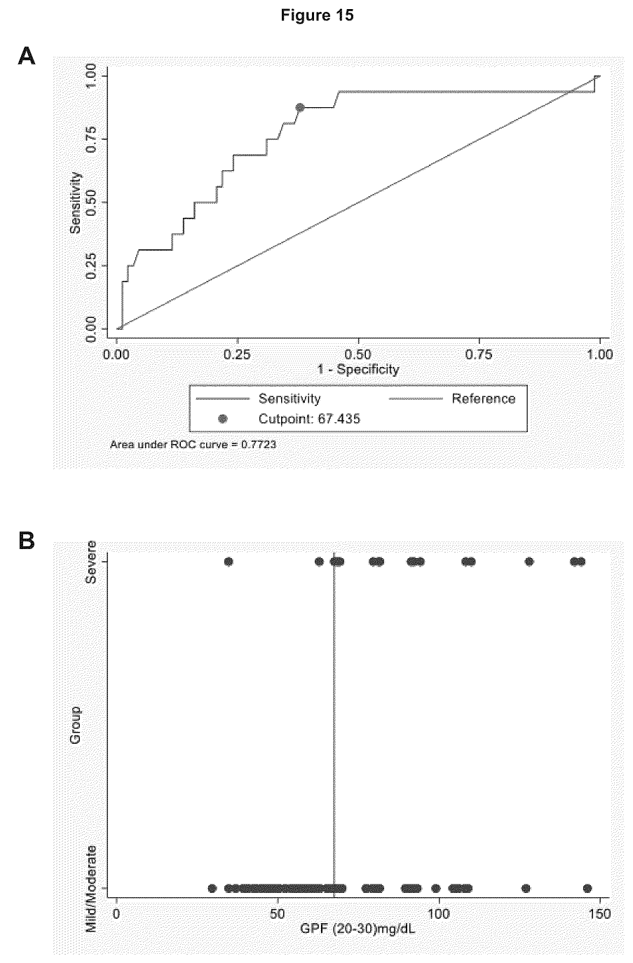

We next examined the association between these biomarkers and COVID-19disease severity. γ′ fibrinogen was significantly associated with disease severity in COVID-19 patients compared to COVID-19-free subjects (p=0.0008) (FIG. 14). FIG. 15A shows a ROC curve for the association between γ′ fibrinogen levels and disease severity. The AUC for γ′ fibrinogen was 0.77 (95% CI: 0.64-0.90). Youden's index (FIG. 15B) showed a cut-point of 67.4 mg/dL between mild/moderate and severe COVID-19patients. The sensitivity at this cut-point was 0.88 and the specificity was 0.62.

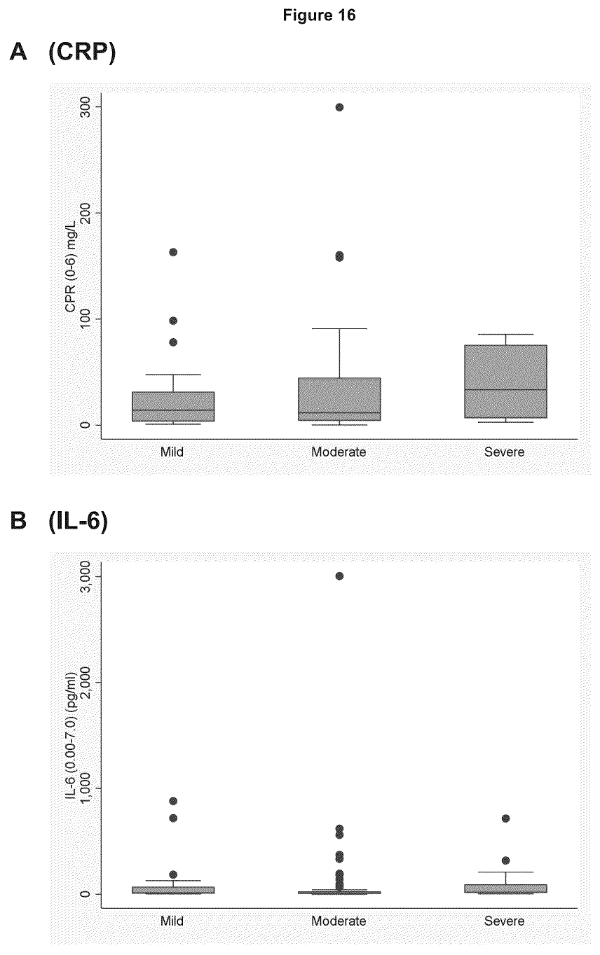

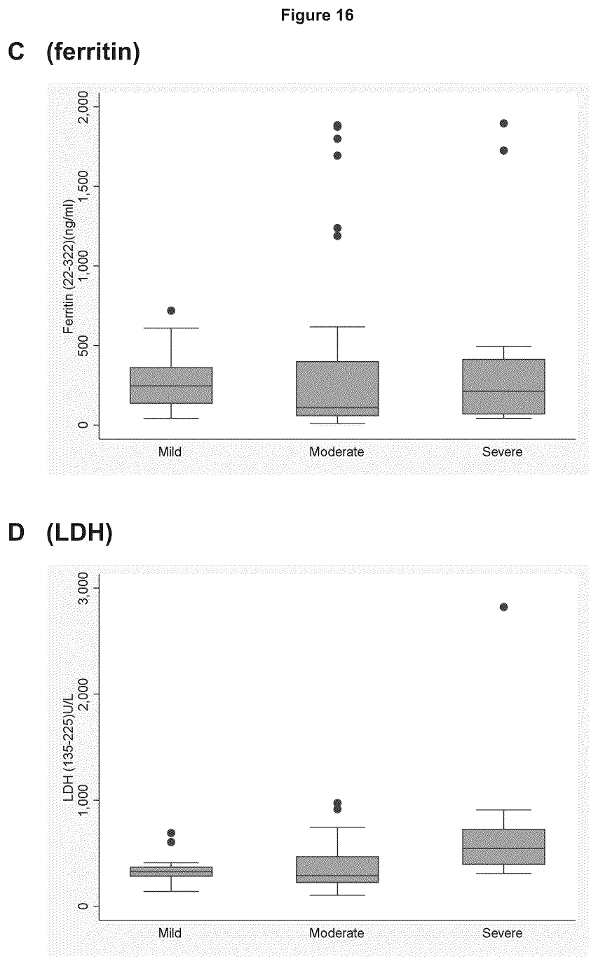

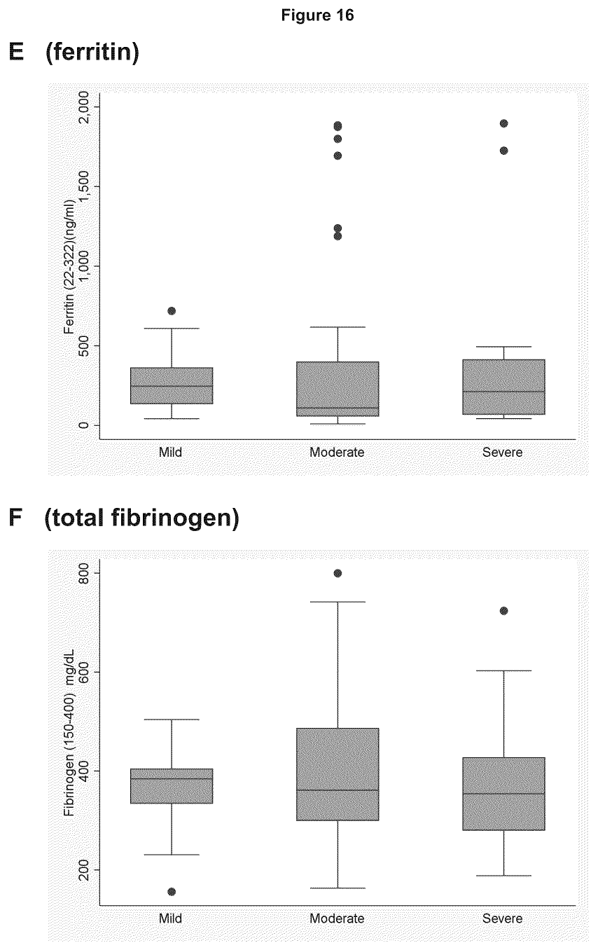

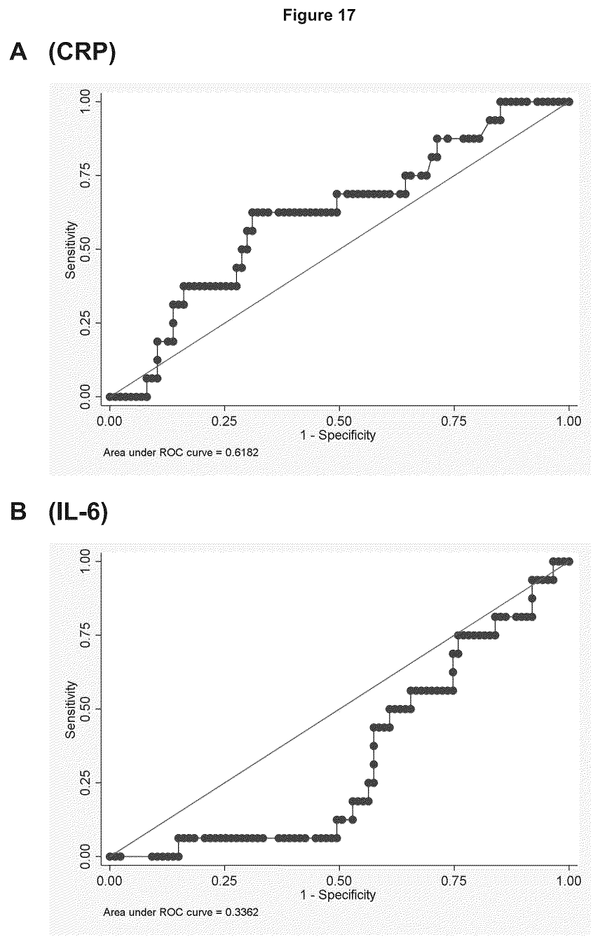

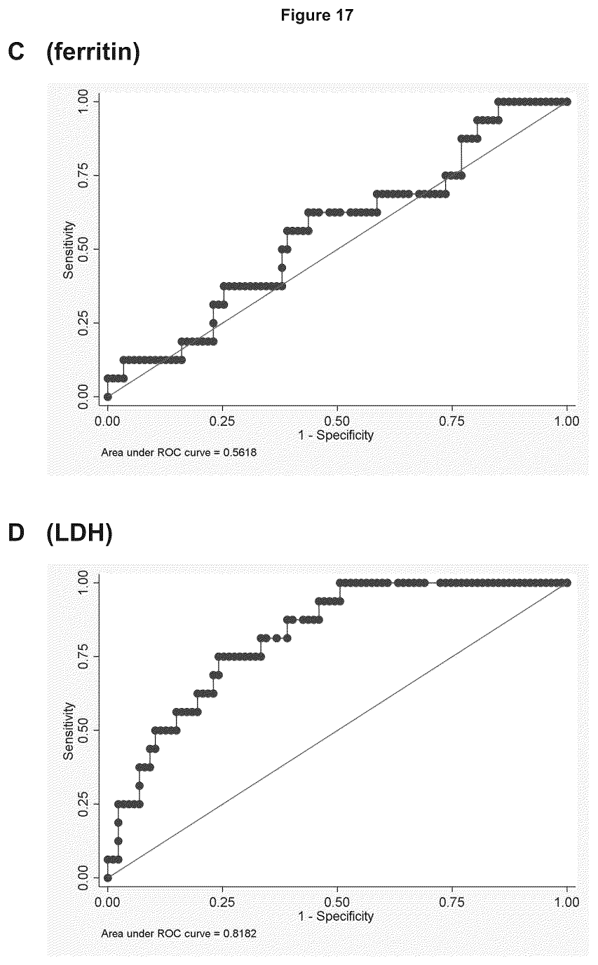

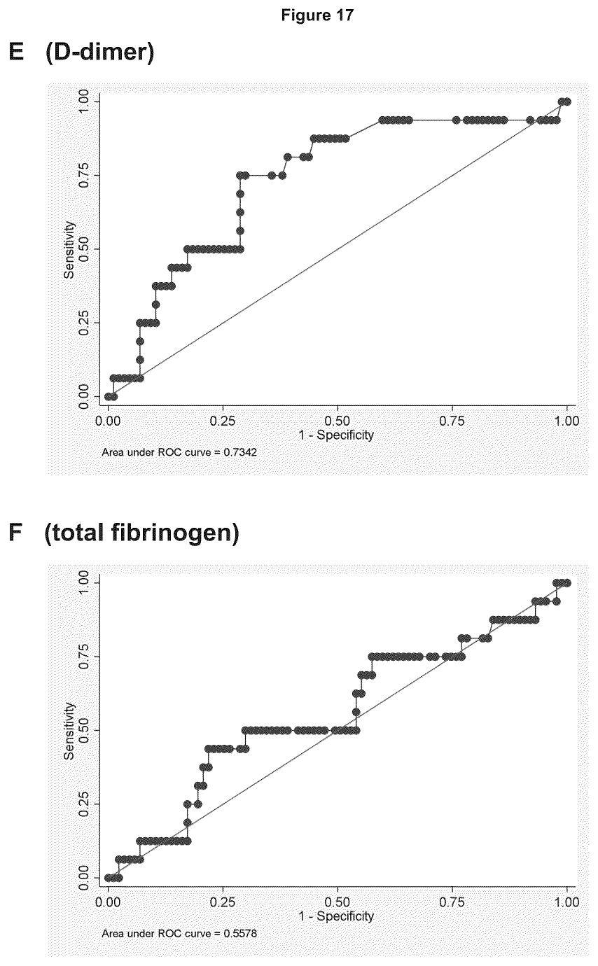

In contrast, CRP levels (FIG. 16A) were not significantly associated with disease severity in COVID-19 patients, and neither were IL-6 levels (FIG. 16B) or ferritin levels (FIG. 16C). ROC curves for CRP showed an AUC of 0.62 (95% CI: 0.47-0.77) (FIG. 17A), ROC curves for IL-6 showed an AUC of 0.34 (95% CI: 0.21-0.46) (FIG. 17B), and ROC curves for ferritin showed an AUC of 0.56 (95% CI: 0.41-0.71) (FIG. 17C). However, LDH levels showed a significant association with disease severity in COVID-19 patients (FIG. 16D), as did D-dimers (FIG. 16E). ROC curves for LDH showed an AUC of 0.82 (95% CI: 0.72-0.91) (FIG. 17D) and ROC curves for D-dimers showed an AUC of 0.73 (95% CI: 0.60-0.87) (FIG. 17E). Total fibrinogen levels did not show a significant association with disease severity in COVID-19 patients (FIG. 16F), with ROC curves showing an AUC of 0.56 (95% CI: 0.39-0.72), again suggesting that total fibrinogen levels were not simply a surrogate for γ′ fibrinogen levels.

### Discussion

Since the identification of the novel coronavirus COVID-19, significant analysis has been made on the already well characterised inflammatory biomarkers D-dimers, IL-6, CRP, ferritin, erythrocyte sedimentation rate (ESR), amongst others. However, γ′ fibrinogen has not previously been considered for usefulness in providing any marker or assessment of COVID-19. The experimental studies conducted by the inventors and described herein showed that when used as a biomarker, γ′ fibrinogen levels are often elevated in COVID-19 patients upon admission to hospital.

These γ′ fibrinogen levels were also shown to be elevated concomitantly with other specific inflammatory markers. In the present experimental studies, the inventors also observed increased concentrations of D-dimers, IL-6, CRP, and ferritin in COVID-19 patients, and this observation was consistent with previous reporting by others.

However, even more importantly, the inventors identified that there was a direct correlation between the levels of γ′ fibrinogen, and the subsequent severity of the disease in the COVID-19 patient as their illness progressed. This was not observed for the other inflammatory markers that were studied. Thus, greater elevation of γ′ fibrinogen levels were found to correlate to a subsequent greater disease severity for a COVID-19 patient. The benefit of this finding therefore enabling assessment of the γ′ fibrinogen levels in a COVID-19 patient to indicate potential severity of their disease progression. This means that COVID-19 patients who are predicted to progress to severe disease outcomes due to assessment of them having higher levels of γ′ fibrinogen, can be prioritized to receive appropriate early treatment, for example ICU admission, in anticipation of severe disease symptoms to give them the best chance of recovery from the disease.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons for conciseness. Inclusion does not constitute an admission is made that any of the references constitute prior art or are part of the common general knowledge of those working in the field to which this invention relates.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

It is to be appreciated that reference to “one example” or “an example” of the invention is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

It is to be understood that the terminology employed above is for the purpose of description and should not be regarded as limiting. The described embodiment is intended to be illustrative of the invention, without limiting the scope thereof. The invention is capable of being practised with various modifications and additions as will readily occur to those skilled in the art.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Various substantially and specifically practical and useful exemplary embodiments of the claimed person matter are described herein, textually and/or graphically, including the best mode, if any, known to the inventors for carrying out the claimed person matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The inventor(s) expects skilled artisans to employ such variations as appropriate, and the inventor(s) intends for the claimed person matter to be practiced other than as specifically described herein. Accordingly, as permitted by law, the claimed person matter includes and covers all equivalents of the claimed person matter and all improvements to the claimed person matter. Moreover, every combination of the above described elements, activities, and all possible variations thereof are encompassed by the claimed person matter unless otherwise clearly indicated herein, clearly and specifically disclaimed, or otherwise clearly contradicted by context.

The present invention is not to be limited in scope by the specific embodiments described herein, which are intended for the purpose of exemplification only. Functionally equivalent products, compositions and methods are clearly within the scope of the invention as described herein.

The use of any and all examples, or exemplary language (e.g., “such as” or “for example”) provided herein, is intended merely to better illuminate one or more embodiments and does not pose a limitation on the scope of any claimed person matter unless otherwise stated. No language in the specification should be construed as indicating any non-claimed person matter as essential to the practice of the claimed person matter.

The use of the terms “a”, “an”, “said”, “the”, and/or similar referents in the context of describing various embodiments (especially in the context of the claimed person matter) are to be construed to cover both the singular and the plural, unless otherwise indicated herein or clearly contradicted by context.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Throughout the specification unless the context requires otherwise, the word “include” or variations such as “includes” or “including”, will be understood to imply the inclusion of a stated integer or group of integers but not the exclusion of any other integer or group of integers.

Moreover, when any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate sub-range defined by such separate values is incorporated into the specification as if it were individually recited herein. For example, if a range of 1 to 10 is described, that range includes all values there between, such as for example, 1.1, 2.5, 3.335, 5, 6.179, 8.9999, etc., and includes all sub-ranges there between, such as for example, 1 to 3.65, 2.8 to 8.14, 1.93 to 9, etc.

Accordingly, every portion (e.g., title, field, background, summary, description, abstract, drawing figure, etc.) of this application, other than the claims themselves, is to be regarded as illustrative in nature, and not as restrictive; and the scope of person matter protected by any patent that issues based on this application is defined only by the claims of that patent.

While there are shown and described presently further embodiments of the application, it is to be distinctly understood that the application is not limited thereto but may be otherwise variously embodied and practiced within the scope of the following claims.

Any of the features of an embodiment of aspects is applicable to all other aspects and embodiments identified herein. Any of the features of an embodiment is independently combinable, partly or wholly with other embodiments described herein in any way, e.g., one, two, or three or more embodiments may be combinable in whole or in part.

1. A method of assessing a COVID-19 infection in a person, the method comprising analysing the concentration or levels of one or more biomarkers in a biological sample from a person, wherein the one or more biomarkers comprises γ′ fibrinogen.

2. A method according to claim 1, wherein analysing the concentration or levels of one or more biomarkers in a biological sample from a person, wherein the one or more biomarkers comprises γ′ fibrinogen, comprises detecting and quantifying the amount of the one or more biomarkers in a blood sample from the person.

3. A method according to claim 1, wherein concentration or levels of γ′ fibrinogen in a biological sample from a person infected with COVID-19 are elevated when compared to a biological sample taken from the person when the person was not infected with COVID-19 or when compared to biological samples taken from one or more other persons not infected with COVID-19.

4. A method according to claim 1, wherein concentration or levels of γ′ fibrinogen is greater than approximately 46 mg/dl in a biological sample from a person infected with COVID-19.

5. A method according to claim 1, wherein analysing the concentration or levels of one or more biomarkers in a biological sample from a person, wherein the one or more biomarkers comprises γ′ fibrinogen, is used for predicting COVID-19 disease severity and/or for making a prognosis of severe COVID-19 disease in a person infected with COVID-19.

6. A method according to claim 5, wherein a concentration of γ′ fibrinogen of between approximately 46 mg/dL and 67 mg/dl in a biological sample from a person indicates a mild to moderate COVID-19 infection in the person.

7. A method according to claim 5, wherein a concentration of γ′ fibrinogen of approximately 67 mg/dL or greater in a biological sample from a person indicates a severe COVID-19 infection in the person.

8. A method according to claim 6, wherein a concentration of γ′ fibrinogen of between approximately 46 mg/dL and 67 mg/dl in the biological sample from the person indicates a mild to moderate COVID-19 infection in the person corresponding to a SpO₂ of above approximately 91% in the person.

9. A method according to claim 7, wherein a concentration of γ′ fibrinogen of approximately 67 mg/dL or greater in the biological sample from the person indicates a severe COVID-19 infection in the person corresponding to a SpO₂ of below approximately 91% in the person.

10. A method according to claim 1, wherein an assay is used in analysing the concentration or levels of the one or more biomarkers in a biological sample from a person, wherein the one or more biomarkers comprises γ′ fibrinogen.

11. A method according to claim 10, wherein the assay comprises an ELISA.

12. A method according to claim 1, wherein the one or more biomarkers further comprise D-Dimer and/or ferritin.

13. A method according to claim 1, wherein the one or more biomarkers further comprise CRP and/or IL-6.

14. A method according to claim 5, wherein COVID-19 disease severity or severe COVID-19 disease comprises ICU admission and/or ventilation for the COVID-19 infected person.

15. A method according to claim 5, wherein COVID-19 disease severity or severe COVID-19 disease comprises a SpO₂ of below approximately 91% in the COVID-19 infected person.

16. A method of treating a person infected with COVID-19 according to the severity of their COVID-19 infection as determined using a method of assessing a COVID-19 infection in a person according to claim 1.

17. A method for prognosis of COVID-19 disease severity in a person comprising a method of assessing a COVID-19 infection in a person according to claim 1.

18. A kit for diagnosing, monitoring, and/or forming a prognosis of disease severity of COVID-19 in a person comprising an assay according to claim 10.

19-32. (canceled)
